(19) United States
(12) Patent Application Publication
(10) Pub. No.: US 2008/0044373 A1
(43) Pub. Date: Feb. 21, 2008

(54) Cosmetic composition for the lips, combining a phosphate surfactant and a silicone polymer

(75) Inventors: Philippe Ilekti, Maisons Alfort (FR); Laure Le Chaux, I'Hay-les-Roses (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/808,713

(22) Filed: Jun. 12, 2007

Related U.S. Application Data
(60) Provisional application No. 60/816,859, filed on Jun. 28, 2006.

(30) Foreign Application Priority Data
Jun. 13, 2006 (FR) 06.52111

(51) Int. Cl. A61K 31/74; A61Q 1/00

(52) U.S. Cl. 424/78.03

(57) ABSTRACT

The present invention relates to a cosmetic composition for the lips, comprising, in a physiologically acceptable medium, at least: one phosphate surfactant, and one silicone polymer comprising at least one unit comprising: (1) polyorganosiloxanes comprising at least two groups capable of establishing hydrogen interactions, these two groups being in the polymer chain, and/or (2) polyorganosiloxanes comprising at least two groups capable of establishing hydrogen interactions, these two groups being on grafts or branches.

This non provisional application claims the benefit of French Application No. 06 52111 filed on Jun. 13, 2006 and U.S. Provisional Application No. 60/816,859 filed on Jun. 28, 2006.

The present invention relates to a cosmetic composition for the lips, comprising at least one phosphate surfactant and a silicone polymer.

It is commonplace in cosmetic or dermatological products to find a structured, i.e. gelled and/or rigidified, liquid phase. This is especially the case in solid compositions, in particular solid cast compositions, lipsticks and lip balms, eyeshadows, concealer products and cast foundations. This structuring is conventionally obtained using waxes or fillers or, more recently, using specific gelling agents.

Thus, documents WO-A-97/36573, U.S. Pat. No. 5,874,069, U.S. Pat. No. 5,919,441, U.S. Pat. No. 6,051,216, WO-A-02/17870, WO-A-02/17871, EP-A-1 177 784, WO-A-99/06473, and U.S. Pat. No. 6,353,076 which is a division of U.S. Pat. No. 6,051,216, propose cosmetic compositions such as deodorant sticks or gels, comprising a silicone oily phase gelled with a silicone polymer of the polysiloxane/polyamide type.

The use of silicone polymers gives access to a cosmetic composition of novel solid texture, i.e. having relatively low rigidity and high elasticity. This texture does not correspond either to that of a conventional stick with relatively high rigidity, or to that of a standard gel whose consistency is liquid or pasty.

In conjunction with this texture adjustment, the inventors have sought to gain access to a composition formulation that also has good deformability qualities for easy and pleasant application and that may also, if necessary, be satisfactory in terms of gloss.

The inventors have found, unexpectedly, that the use of a phosphate surfactant in combination with such a silicone polymer can produce compositions that are satisfactory in these terms.

Consequently, according to a first aspect, the present invention relates to a cosmetic composition for the lips, comprising, in a physiologically acceptable medium, at least:

one phosphate surfactant, and

one silicone polymer comprising at least one unit comprising:

(1) polyorganosiloxanes comprising at least two groups capable of establishing hydrogen interactions, these two groups being in the polymer chain, and/or

(2) polyorganosiloxanes comprising at least two groups capable of establishing hydrogen interactions, these two groups being on grafts or branches.

In one exemplary embodiment, the compositions according to the invention are in solid form.

The invention allows the production of compositions with improved glidance that are pleasant to apply. The deposit on the lips is homogeneous.

A subject of the present invention is also a lip makeup process in which a composition as defined above is applied to the lips.

According to another of its aspects, the invention also relates to the use of a silicone polymer in combination with a phosphate surfactant for preparing a cosmetic composition for the lips, capable of producing a film of improved gloss.

According to another of its aspects, the invention relates to a process for preparing a cosmetic composition for the lips, capable of producing a film of improved gloss comprising the step of combining a silicone polymer. According to another of its aspects, the invention relates to the use of a silicone polymer in combination with a phosphate surfactant for producing a uniform and stabilized dispersion of solid particles, for instance pigments, in a cosmetic composition for the lips.

According to another of its aspects, the invention relates to a process for producing a uniform and stabilized dispersion of solid particles, for instance pigments, in a cosmetic composition for the lips comprising the step of combining a silicone polymer.

Although also compatible with a solid formulation like a stick, or a fluid formulation like a gloss, the compositions according to the invention prove to be particularly useful for producing a formulation simultaneously having supple and elastic properties.

The compositions according to the invention thus prove, unexpectedly, to have a texture that may be associated with a particular application gesture.

For example, the supple and elastic solid textures obtained according to the invention may be compatible with direct application to the lips without requiring the use of an applicator as in the case of fluid textures. The compositions according to the invention also show, during their application, suppleness, softness and very good elasticity, thus preserving the product for a future application. The product can be deformed non-definitively and regain its initial form after application.

In the form of a dome, the compositions in accordance with the invention advantageously show the behaviour of a deformable and supple elastic solid, giving noteworthy softness on application.

Besides the abovementioned advantages, the combination under consideration in the invention, i.e. phosphate surfactant and silicone polymer, proves to be particularly advantageous for uniformly dispersing particles and especially pigments within the cosmetic compositions containing them. Furthermore, the compositions according to the invention are also protected from exudation phenomena, which are quite obviously undesirable.

Characterization of the Elasticity and the Hardness

In one exemplary embodiment, the compositions according to the invention have a hardness ranging from 10 to 250 g and/or an elasticity of greater than 80%.

The hardness and the elasticity of the composition according to the invention may be measured using a texturometer, which makes it possible to obtain the variation of the resistance to deformation of the composition as a function of the displacement of a spindle into a sample of the said composition.

The texturometer measures the force of resistance to deformation of the composition once the spindle comes into contact with the sample. After reaching a programmed maximum depth L0 into the sample, the spindle returns to the initial point.

The hardness (expressed in grams or in newtons) is equal to the resistance value of the composition when the spindle is at the end of its course, and the elasticity (expressed as a percentage) is equal to the ratio of i) the distance L at which there is breaking of contact between the spindle and the sample during the withdrawal of the spindle and of ii) the distance L0. The breaking of contact is reflected by the disappearance of the resistance force of the composition on the spindle.

The elasticity is proportional to the distance over which the system “accompanies” the return of the spindle: the greater the value, the more elastic the system.

The texturometer used may especially be a Stable Micro System TAX-T2i® texturometer equipped with operating software such as Texture Expert Exceed ® and fitted with a P/0.5 HS Rheo plastic hemispherical spindle.

The applied parameters are for example the following:

speed before contact: 0.1 mm.s⁻¹,

displacement speed in the sample: 0.1 mm.s⁻¹,

withdrawal speed: 0.1 mm.s⁻¹,

maximum depth L0: 1 mm.

The composition samples are prepared by hot-casting a sufficient amount of the test composition, for example into a pretared 100×15 mm Petri dish, to obtain a sample about 1 cm thick. The advantage of choosing this conditioning is its width, which is sufficient to overcome any edge effect. Two Petri dishes are thus prepared and are left to stand for a minimum of 24 hours at 20° C. before characterization.

A minimum of three measurements are taken on each sample: one at the centre and others at points equidistant from the centre and the edge of the dish.

The hardness and the elasticity are equal to the mean of the measurements taken, which are a minimum number of six.

The hardness of the composition according to the invention is such that the composition is self-supporting and can be broken down easily to form a satisfactory deposit on the skin and the lips. In addition, with this hardness, the composition of the invention shows good resistance to impacts.

In one exemplary embodiment, the hardness may range from 30 g to 200 g, for example from 50 g to 190 g, or even from 70 to 175 g, or for example from 100 g to 150 g.

The compositions according to the invention may have an elasticity of greater than 90% and in particular close to 95%.

Silicone Polymer

As indicated previously, the compositions according to the invention comprise at least one silicone polymer.

In one embodiment, the silicone polymers of the composition are preferably solid at room temperature (25° C.) and atmospheric pressure (760 mmHg).

For the purposes of the invention, the term “polymer” means a compound containing at least 2 repeating units, preferably at least 3 repeating units and better still 10 repeating units.

The silicone polymers of the composition of the invention are polymers of the polyorganosiloxane type, for instance those described in documents U.S. Pat. No. 5,874,069, U.S. Pat. No. 5,919,441, U.S. Pat. No. 6,051,216 and U.S. Pat. No. 5,981,680.

According to the invention, the silicone polymers may belong to the following two families:

(1) polyorganosiloxanes comprising at least two groups capable of establishing hydrogen interactions, these two groups being located in the polymer chain, and/or

(2) polyorganosiloxanes comprising at least two groups capable of establishing hydrogen interactions, these two groups being located on grafts or branches.

The groups capable of establishing hydrogen interactions may be chosen from ester, amide, sulfonamide, carbamate, thiocarbamate, urea, urethane, thiourea, oxamido, guanidino and biguanidino groups, and combinations thereof.

A) According to a first variant, the silicone polymers are polyorganosiloxanes as defined above in which the units capable of establishing hydrogen interactions are located in the polymer chain.

The silicone polymers may be more particularly polymers comprising at least one unit corresponding to the general formula I: 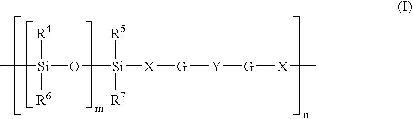
in which:

1) R⁴, R⁵, R⁶ and R⁷, which may be identical or different, represent a group chosen from:

linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,

C₆ to C₁₀ aryl groups, optionally substituted with one or more C₁ to C₄ alkyl groups,

polyorganosiloxane chains possibly containing one or more oxygen, sulfur and/or nitrogen atoms;

2) the groups X, which may be identical or different, represent a linear or branched C₁ to C₃₀ alkylenediyl group, possibly containing in its chain one or more oxygen and/or nitrogen atoms;

3) Y is a saturated or unsaturated, C₁ to C₅₀ linear or branched divalent alkylene, arylene, cycloalkylene, alkylarylene or arylalkylene group, possibly comprising one or more oxygen, sulfur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups; or

4) Y represents a group corresponding to the formula: 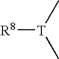
in which

T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and

R⁸ represents a linear or branched C₁ to C₅₀ alkyl group or a polyorganosiloxane chain, possibly comprising one or more ester, amide, urethane, thiocarbamate, urea, thiourea and/or sulfonamide groups, which may possibly be linked to another chain of the polymer;

5) the groups G, which may be identical or different, represent divalent groups chosen from: 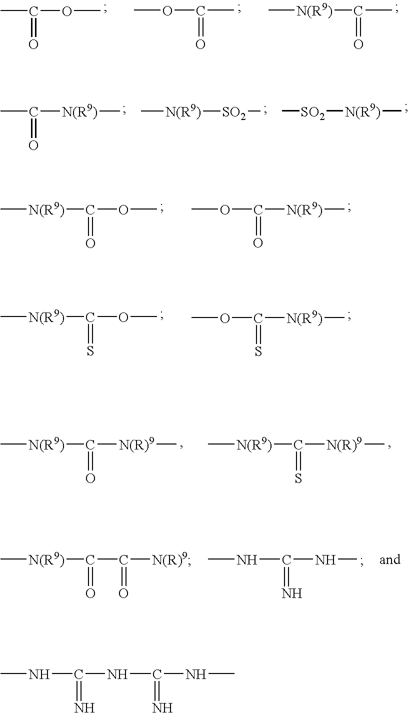
in which R⁹ represents a hydrogen atom or a linear or branched C₁ to C₂₀ alkyl group, on condition that at least 50% of the groups R⁹ of the polymer represent a hydrogen atom and that at least two of the groups G of the polymer are a group other than:

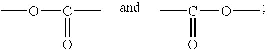

6) n is an integer ranging from 2 to 500 and preferably from 2 to 200, and m is an integer ranging from 1 to 1000, preferably from 1 to 700 and better still from 6 to 200.

In one embodiment of the invention, 80% of the groups R⁴, R⁵, R⁶ and R⁷ of the polymer may be chosen from methyl, ethyl, phenyl and 3,3,3-trifluoropropyl groups.

In one embodiment of the invention, Y may represent various divalent groups, furthermore optionally comprising one or two free valencies to establish bonds with other units of the polymer or copolymer. For example, Y may represent a group chosen from:

a) linear C₁ to C₂₀ and preferably C₁ to C₁₀ alkylene groups,

b) C₃₀ to C₅₆ branched alkylene groups possibly comprising rings and unconjugated unsaturations,

c) C₅-C₆ cycloalkylene groups,

d) phenylene groups optionally substituted with one or more C₁ to C₄₀ alkyl groups,

e) C₁ to C₂₀ alkylene groups comprising from 1 to 5 amide groups,

f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups,

g) polyorganosiloxane chains of formula: 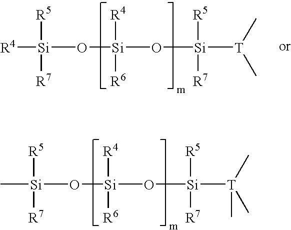
in which R⁴, R⁵, R⁶, R⁷, T and m are as defined above.

B) According to the second variant, the polyorganosiloxanes may be polymers comprising at least one unit corresponding to formula (II): 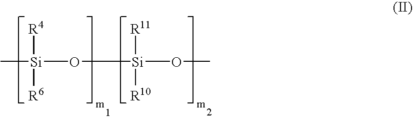
in which

R⁴ and R⁶, which may be identical or different, are as defined above for formula (I),

R¹⁰ represents a group as defined above for R⁴ and R⁶, or represents a group of formula —X-G-R¹² in which X and G are as defined above for formula (I) and R¹² represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,

R¹¹ represents a group of formula —X-G-R¹² in which X, G and R¹² are as defined above,

m₁ is an integer ranging from 1 to 998, and

m₂ is an integer ranging from 2 to 500.

In one embodiment of the invention, the silicone polymer may be a homopolymer, that is to say a polymer comprising several identical units, in particular units of formula (I) or of formula (II).

In one embodiment of the invention, it is also possible to use a polymer consisting of a copolymer comprising several different units of formula (I), that is to say a polymer in which at least one of the groups R⁴, R⁵, R⁶, R⁷, X, G, Y, m and n is different in one of the units. The copolymer may also be formed from several units of formula (II), in which at least one of the groups R⁴, R⁶, R¹⁰, R¹¹, m₁ and m₂ is different in at least one of the units.

In an another embodiment of the invention is also possible to use a polymer comprising at least one unit of formula (I) and at least one unit of formula (II), the units of formula (I) and the units of formula (II) possibly being identical to or different from each other.

According to one variant of the invention, it is also possible to use a polymer furthermore comprising at least one hydrocarbon-based unit comprising two groups capable of establishing hydrogen interactions, chosen from ester, amide, sulfonamide, carbamate, thiocarbamate, urea, urethane, thiourea, oxamido, guanidino and biguanidino groups, and combinations thereof.

These copolymers may be block polymers or grafted polymers.

According to one embodiment of the invention, the groups capable of establishing hydrogen interactions may be amide groups of formulae —C(O)NH— and —HN—C(O)—.

In this case, the silicone polymer may be a polymer comprising at least one unit of formula (III) or (IV): 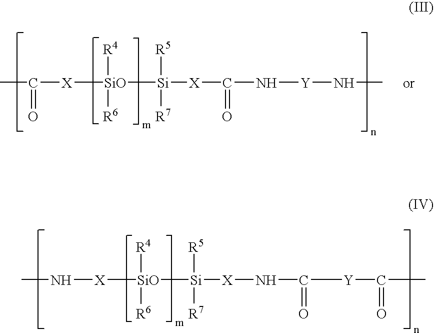
in which R⁴, R⁵, R⁶, R⁷, X, Y, m and n are as defined above.

Such a unit may be obtained:

either by a condensation reaction between a silicone containing α,ω-carboxylic acid ends and one or more diamines, according to the following reaction scheme:

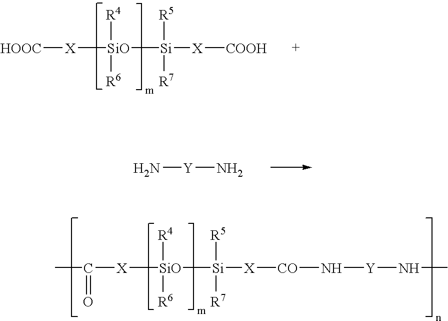

or by reaction of two molecules of α-unsaturated carboxylic acid with a diamine according to the following reaction scheme:
CH₂═CH—X¹ —COOH+H₂N—Y—NH₂→CH₂═CH—X¹ —CO—NH—Y—NH—CO—X¹ —CH′CH₂
followed by the addition of a siloxane to the ethylenic unsaturations, according to the following scheme: 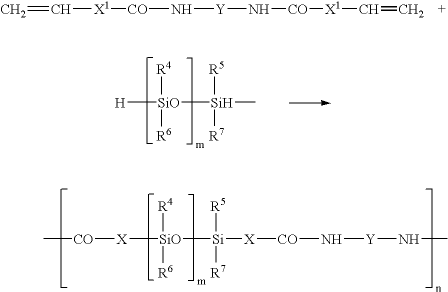
in which X¹—(CH₂)₂— corresponds to X defined above and Y, R⁴, R⁵, R⁶, R⁷ and m are as defined above;

or by reaction of a silicone containing α,ω-NH₂ ends and a diacid of formula HOOC—Y—COOH according to the following reaction scheme:

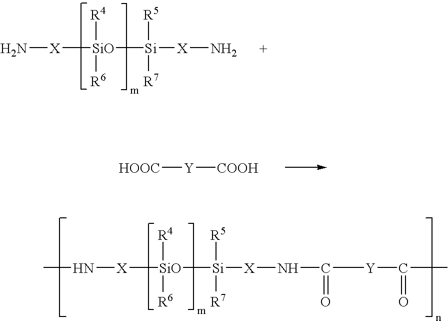

In these polyamides of formula (III) or (IV), m may be in the range from 1 to 700, for example from 15 to 500, or for example from 5 to 200, and n may be in the range from 1 to 500, for example from 1 to 100 or for example from 4 to 25,

X may be a linear or branched alkylene chain containing from 1 to 30 carbon atoms, for example from 1 to 20 carbon atoms, for example from 5 to 15 carbon atoms, or for example 10 carbon atoms, and

Y may be an alkylene chain that is linear or branched or that possibly comprises rings and/or unsaturations, containing from 1 to 40 carbon atoms, for example from 1 to 20 carbon atoms, or for example from 2 to 6 carbon atoms, or for example 6 carbon atoms.

In formulae (III) and (IV), the alkylene group representing X or Y may optionally contain in its alkylene part at least one of the following components:

1) one to five amide, urea, urethane or carbamate groups,

2) a C₅ or C₆ cycloalkyl group, and

3) a phenylene group optionally substituted with 1 to 3 identical or different C₁ to C₃ alkyl groups.

In formulae (III) and (IV), the alkylene groups may also be substituted with at least one component chosen from the group consisting of:

a hydroxyl group,

a C₃ to C₈ cycloalkyl group,

one to three C₁ to C₄₀ alkyl groups,

a phenyl group optionally substituted with one to three C₁ to C₃ alkyl groups,

a C₁ to C₃ hydroxyalkyl group, and

a C₁ to C₆ aminoalkyl group.

In these formulae (III) and (IV), Y may also represent: 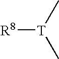
in which R⁸ represents a polyorganosiloxane chain and T represents a group of formula: 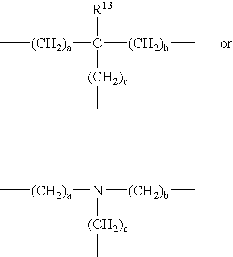
in which a, b and c are, independently, integers ranging from 1 to 10, and R¹³ is a hydrogen atom or a group such as those defined for R⁴, R⁵, R⁶ and R⁷.

In formulae (III) and (IV), R⁴, R⁵, R⁶ and R⁷ may represent, independently, a linear or branched C₁ to C₄₀ alkyl group, for example a CH₃, C₂H₅, n-C₃H₇ or isopropyl group, a polyorganosiloxane chain or a phenyl group optionally substituted with one to three methyl or ethyl groups.

As has been seen previously, the polymer may comprise identical or different units of formula (III) or (IV).

Thus, the polymer may be a polyamide containing several units of formula (III) or (IV) of different lengths, i.e. a polyamide corresponding to formula (V):

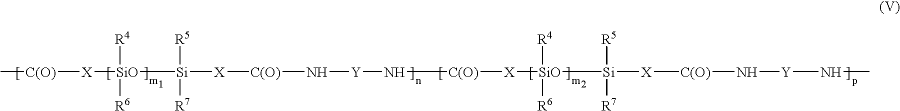

in which X, Y, n and R⁴ to R⁷ have the meanings given above, m₁ and m₂, which are different, are chosen in the range from 1 to 1000, and p is an integer ranging from 2 to 300.

In this formula, the units may be structured to form either a block copolymer, or a random copolymer or an alternating copolymer. In this copolymer, the units may be not only of different lengths, but also of different chemical structures, for example containing different groups Y. In this case, the polymer may correspond to formula VI: 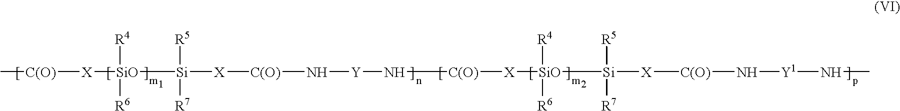
in which R⁴ to R⁷, X, Y, m₁, m₂, n and p have the meanings given above and Y¹ is different from Y but chosen from the groups defined for Y. As previously, the various units may be structured to form either a block copolymer, or a random copolymer or an alternating copolymer.

In this first embodiment of the invention, the silicone polymer may also consist of a grafted copolymer. Thus, the polyamide containing silicone units may be grafted and optionally crosslinked with silicone chains containing amide groups. Such polymers may be synthesized with trifunctional amines.

In this case, the polymer may comprise at least one unit of formula (VII): 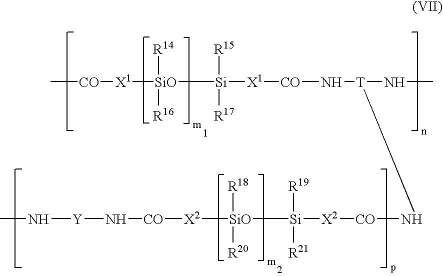
in which X¹ and X², which are identical or different, have the meaning given for X in formula (I), n is as defined in formula (I), Y and T are as defined in formula (I), R¹⁴ to R²¹ are groups chosen from the same group as R⁴ to R⁷, m₁ and m₂ are numbers in the range from 1 to 1000, and p is an integer ranging from 2 to 500.

In formula (VII), it may be possible that:

p is in the range from 1 to 25 and better still from 1 to 7,

R¹⁴ to R²¹ are methyl groups,

T corresponds to one of the following formulae: 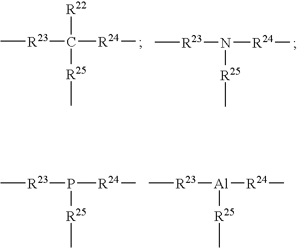
in which R²² is a hydrogen atom or a group chosen from the groups defined above for R⁴ to R⁷, and R²³, R²⁴ and R²⁵ are, independently, linear or branched alkylene groups, or for example correspond to the formula: 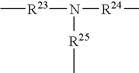
with R²³, R²⁴ and R²⁵ representing —CH₂—CH₂—,

m₁ and m₂ are in the range from 15 to 500, or for example from 15 to 45,

X¹ and X² represent —(CH₂)₁₀—, and

Y represents —CH₂—.

These polyamides containing a grafted silicone unit of formula (VII) may be copolymerized with polyamide-silicones of formula (II) to form block copolymers, alternating copolymers or random copolymers. The weight percentage of grafted silicone units (VII) in the copolymer may range from 0.5% to 30% by weight.

According to the invention, as has been seen previously, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to one embodiment variant of the invention, a copolymer of silicone polyamide and of hydrocarbon-based polyamide, or a copolymer comprising units of formula (III) or (IV) and hydrocarbon-based polyamide units, may be used. In this case, the polyamide-silicone units may be located at the ends of the hydrocarbon-based polyamide.

In one embodiment, the composition according to the invention may comprise at least one polydimethylsiloxane block polymer of general formula (I) with an index m of greater than 50, for example greater than 75, or for example greater than 100, or for example of about 120.

According to one embodiment, the composition may comprise at least one polydimethylsiloxane block polymer of general formula (I) with an index m of about 15.

In one embodiment, the composition according to the invention may comprise:

at least one first polymer comprising at least one unit of general formula (I) which m ranges from 50 to 600, for example from 60 to 400, or for example from 75 to 200, or for example is about 120, and

at least one second polymer comprising at least one unit of general formula (I) in which m ranges from 5 to 100, for example from 10 to 75, or for example is about 15.

In one embodiment, the composition according to the invention may comprise:

at least one first polymer comprising at least one unit of formula (III) in which m ranges from 50 to 600, for example from 60 to 400, or for example from 75 to 200, or for example is about 120, and

at least one second polymer comprising at least one unit of formula (III) in which m ranges from 5 to 100 and for example from 10 to 75, or for example is about 15.

Thus, a subject of the invention is also a cosmetic composition for the lips, comprising, in a physiologically acceptable medium, at least one first silicone polymer and at least one second silicone polymer, each independently comprising at least one unit of formula (III) as defined above, in which:

m ranges from 50 to 600, for example from 60 to 400, or for example from 75 to 200, or for example is about 120 for the first polymer, and

m ranges from 5 to 100 and for example from 10 to 75, or for example is about 15 for the second polymer.

In one embodiment, these first and second polymers correspond to formula (III) in which R⁴, R⁵, R⁶ and R⁷ independently represent a linear or branched C₁-C₄₀ alkyl group, for example a CH₃, C₂H₅, n-C₃H₇ or isopropyl group.

In one embodiment, X and Y may independently represent a group chosen from linear C₁-C₂₀, for example C₁-C₁₀ alkylene groups.

In one embodiment, the first polymer may have a weight-average molecular mass of between 1000 and 500 000 g/mol, for example between 10 000 and 300 000 g/mol and the second polymer may have a weight-average molecular mass ranging from 50 000 to 500 000 g/mol.

In one embodiment, the ratio between the first polymer and the second polymer may range from 5/95 to 95/5, for example from 70/30 to 30/70.

As examples of first polymers that may be used, mention may be made of one of the silicone polyamides obtained in accordance with Examples 1 to 3 of document U.S. Pat. No. 5,981,680.

According to one embodiment variant of the invention, the polymer consists of a homopolymer or copolymer comprising urethane or urea groups. These polymers are described in detail in patent application WO 2003/106 614.

As previously, such a polymer may comprise polyorganosiloxane units containing two or more urethane and/or urea groups, either in the backbone of the polymer or on side chains or as pendent groups.

The polymers comprising at least two urethane and/or urea groups in the backbone may be polymers comprising at least one unit corresponding to formula (VIII) below: 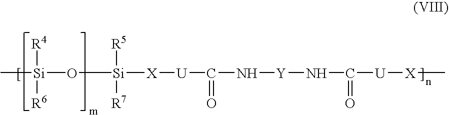
in which R⁴, R⁵, R⁶, R⁷, X, Y, m and n have the meanings given above for formula (I), and U represents —O— or —NH—, such that: 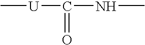
corresponds to a urethane or urea group.

In this formula (VIII), Y may be a linear or branched C₁ to C₄₀ alkylene group, optionally substituted with a C₁ to C₁₅ alkyl group or a C₅ to C₁₀ aryl group. In one embodiment, a —(CH₂)₆— group is used.

Y may also represent a C₅ to C₁₂ cycloaliphatic or aromatic group that may be substituted with a C₁ to C₁₅ alkyl group or a C₅ to C₁₀ aryl group, for example a radical chosen from the methylene-4,4-biscyclohexyl radical, the radical derived from isophorone diisocyanate, 2,4- and 2,6-tolylenes, 1,5-naphthylene, p-phenylene and 4,4′-biphenylenemethane. Generally, it is preferred for Y to represent a linear or branched C₁ to C₄₀ alkylene radical or a C₄ to C₁₂ cycloalkylene radical.

Y may also represent a polyurethane or polyurea block corresponding to the condensation of several diisocyanate molecules with one or more molecules of coupling agents of the diol or diamine type. In this case, Y comprises several urethane or urea groups in the alkylene chain.

It may correspond to formula (IX): 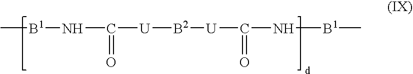
in which B¹ is a group chosen from the groups given above for Y, U is —O— or —NH— and B² is chosen from:

linear or branched C₁ to C₄₀ alkylene groups,

C₅ to C₁₂ cycloalkylene groups, optionally bearing alkyl substituents, for example one to three methyl or ethyl groups, or alkylene, for example the diol radical: cyclohexanedimethanol, phenylene groups that may optionally bear C₁ to C₃ alkyl substituents, and

groups of formula: 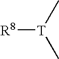
in which T is a hydrocarbon-based trivalent radical possibly containing one or more heteroatoms such as oxygen, sulfur and nitrogen and R⁸ is a polyorganosiloxane chain or a linear or branched C₁ to C₅₀ alkyl chain.

T can represent, for example: 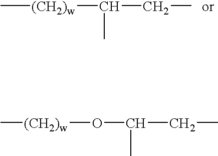
with w being an integer ranging from 1 to 10 and R⁸ being a polyorganosiloxane chain.

When Y is a linear or branched C₁ to C₄₀ alkylene group, the —(CH₂)₂— and —(CH₂)₆— groups are preferred.

In the formula given above for Y, d may be an integer ranging from 0 to 5, preferably from 0 to 3 and more preferably equal to 1 or 2.

Preferably, B² is a linear or branched C₁ to C₄₀ alkylene group, in particular —(CH₂)₂— or —(CH₂)₆— or a group:

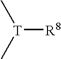

with R⁸ being a polyorganosiloxane chain.

As previously, the silicone polymer may be formed from silicone urethane and/or silicone urea units of different length and/or constitution, and may be in the form of block or random copolymers.

The polymers of formula (VIII) comprising urea or urethane groups in the chain of the silicone polymer may be obtained by reaction between a silicone containing α,ω-NH₂ or —OH end groups, of formula: 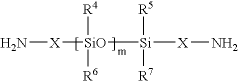
in which m, R⁴, R⁵, R⁶, R⁷ and X are as defined for formula (I), and a diisocyanate OCN—Y—NCO in which Y has the meaning given in formula (I); and optionally a diol or diamine coupling agent of formula H₂N—B²—NH₂ or HO—B²—OH, in which B² is as defined in formula (IX).

According to the stoichiometric proportions between the two reagents, diisocyanate and coupling agent, Y may have the formula (IX) with d equal to 0 or d equal to 1 to 5.

As in the case of the polyamide silicones of formula (IV), (II) or (III), it is possible to use in the invention polyurethane or polyurea silicones containing units of different length and structure, in particular units whose lengths differ by the number of silicone units. In this case, the copolymer may correspond, for example, to the formula: 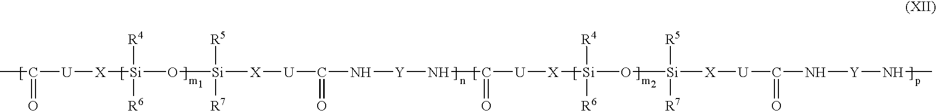
in which R⁴, R⁵, R⁶, R⁷, X, Y and U are as defined for formula (VIII) and m₁, m₂, n and p are as defined for formula (V).

According to the invention, the silicone may also comprise urethane and/or urea groups no longer in the backbone but as side branches.

In this case, the polymer may comprise at least one unit of formula: 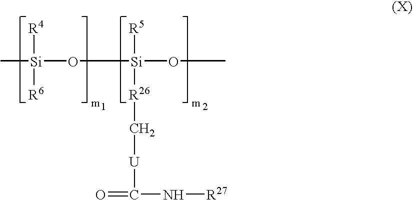
in which R⁴, R⁶, R⁵, m₁ and m₂ have the meanings given above for formula (II), and R⁵ for formula (I),

U represents O or NH,

R²⁶ represents a C₁ to C₄₀ alkylene group, optionally comprising one or more heteroatoms chosen from O and N, or a phenylene group, and

R²⁷ is chosen from linear, branched or cyclic, saturated or unsaturated C₁ to C₅₀ alkyl groups, and phenyl groups optionally substituted with one to three C₁ to C₃ alkyl groups.

The polymers comprising at least one unit of formula (X) contain siloxane units and urea or urethane groups, and they may be used as silicone polymer in the compositions of the invention.

The siloxane polymers may have a single urea or urethane group per branch or may have branches containing two urea or urethane groups, or alternatively they may contain a mixture of branches containing one urea or urethane group and branches containing two urea or urethane groups.

They may be obtained from branched polysiloxanes, comprising one or two amino groups per branch, by reacting these polysiloxanes with monoisocyanates.

As examples of starting polymers of this type containing amino and diamino branches, mention may be made of the polymers corresponding to the following formulae:

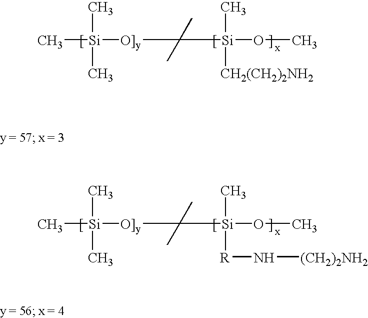

In these formulae, the symbol “/” indicates that the segments may be of different lengths and in a random order, and R represents a linear aliphatic group preferably containing 1 to 6 carbon atoms and better still 1 to 3 carbon atoms.

Such polymers containing branches may be formed by reacting a siloxane polymer, containing at least three amino groups per polymer molecule, with a compound containing only one monofunctional group (for example an acid, an isocyanate or an isothiocyanate) to react this monofunctional group with one of the amino groups and to form groups capable of establishing hydrogen interactions. The amino groups may be on side chains extending from the main chain of the siloxane polymer, such that the groups capable of establishing hydrogen interactions are formed on these side chains, or alternatively the amino groups may be at the ends of the main chain, such that the groups capable of hydrogen interaction will be end groups of the polymer.

As a procedure for forming a polymer containing siloxane units and groups capable of establishing hydrogen interactions, mention may be made of the reaction of a siloxane diamine and of a diisocyanate in a silicone solvent so as to provide a gel directly. The reaction may be performed in a silicone fluid, the resulting product being dissolved in the silicone fluid, at high temperature, the temperature of the system then being reduced to form the gel.

The polymers that are preferred for incorporation into the compositions according to the present invention are siloxane-urea copolymers that are linear and that contain urea groups as groups capable of establishing hydrogen interactions in the backbone of the polymer.

As an illustration of a polysiloxane ending with four urea groups, mention may be made of the polymer of formula: 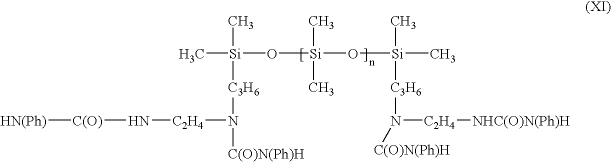
in which Ph is a phenyl group and n is a number from 0 to 300 and in particular from 0 to 100, for example 50.

This polymer is obtained by reacting the following polysiloxane containing amino groups: 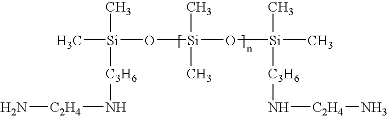
in which n is as defined for formula (XI),
with phenyl isocyanate.

Branched polyurethane or polyurea silicones may also be obtained by using, instead of the diisocyanate OCN—Y—NCO, a triisocyanate of formula:

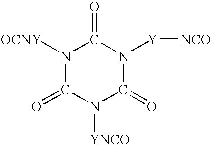

A polyurethane or polyurea silicone containing branches comprising an organosiloxane chain with groups capable of establishing hydrogen interactions is thus obtained. Such a polymer comprises, for example, a unit corresponding to the formula: 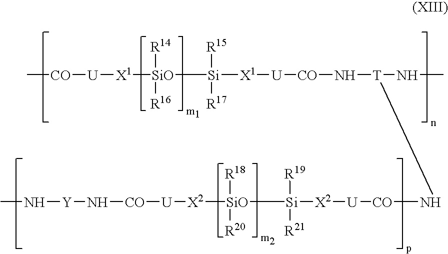
in which X¹ and X², which are identical or different, have the meaning given for X in formula (I), n is as defined in formula (I), Y and T are as defined in formula (I), R¹⁴ to R²¹ are groups chosen from the same group as R⁴ to R⁷, m₁ and m₂ are numbers in the range from 1 to 1000, and p is an integer ranging from 2 to 500.

As in the case of the polyamides, this copolymer can also comprise polyurethane silicone units without branches.

The siloxane-based polyureas and polyurethanes that may be used are:

polymers of formula (VIII) in which m ranges from 50 to 600, for example from 60 to 400, or for example from 75 to 200, or for example is about 100;

mixtures of polymers of formula (VIII) combining:

1) 80% to 99% by weight of a polymer of formula (VIII) in which m ranges from 50 to 600, for example from 60 to 400, or for example from 75 to 200, or for example is about 100, and

2) 1% to 20% of a polymer of formula (VIII) in which m is in the range from 5 to 100, for example from 10 to 75, or for example is about 15;

mixtures of polymers of formula (VIII) combining:

1) 80% to 99% by weight of polymers of formula (VIII) in which n is equal to 2 to 10, or for example from 3 to 6, and

2) 1% to 20% of polymers of formula (VIII) in which n is in the range from 5 to 500, or for example from 30 to 100;

mixtures of polymers of formula (VIII) combining:

1) 1% to 20% by weight of polymers of formula (VIII) in which n is equal to 2 to 10, or for example from 3 to 6, and

2) 80% to 99% of polymers of formula (VIII) in which n is in the range from 30 to 500, or for example from 30 to 100;

polymers of formula (VIII) in which X represents a C₃ to C₁₅, for example a C₅ to C₁₂ or for example a CIO alkyl radical, and

polymers of formula (VIII) in which Y represents a C₃ to C₁₀, for example a C₄ to C₈ or for example a C₆ alkyl radical.

As in the case of the polyamides, copolymers of polyurethane or polyurea silicone and of hydrocarbon-based polyurethane or polyurea may be used in the invention by performing the reaction for synthesizing the polymer in the presence of an α,ω-difunctional block of non-silicone nature, for example a polyester, a polyether or a polyolefin.

As has been seen previously, the silicone polymers of the invention may contain siloxane units in the main chain of the polymer and groups capable of establishing hydrogen interactions, either in the main chain of the polymer or at the ends thereof, or on side chains or branches of the main chain. This may correspond to the following five arrangements: 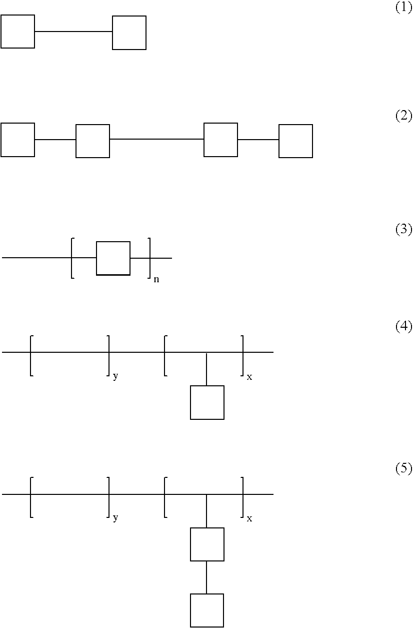
in which the continuous line is the main chain of the siloxane polymer and the squares represent the groups capable of establishing hydrogen interactions.

In case (1), the groups capable of establishing hydrogen interactions are located at the ends of the main chain. In case (2), two groups capable of establishing hydrogen interactions are located at each of the ends of the main chain.

In case (3), the groups capable of establishing hydrogen interactions are located within the main chain in repeating units.

In cases (4) and (5), these are copolymers in which the groups capable of establishing hydrogen interactions are located on branches of the main chain of a first series of units that are copolymerized with units not comprising groups capable of establishing hydrogen interactions.

The polymers and copolymers used in the composition of the invention may have a transition temperature from the solid state to the liquid state ranging from 45° C. to 190° C. They may have a transition temperature from the solid state to the liquid state ranging from 70 to 130° C., or for example from 80° C. to 105° C.

The silicone polymer(s) may be present in the composition according to the invention in a total content ranging from 0.5% to 70% by weight, for example ranging from 5% to 50% by weight, or for example ranging from 10% to 45% by weight, relative to the total weight of the composition.

Phosphate Surfactant

The composition according to the invention comprises at least one phosphate surfactant.

The presence of a phosphate surfactant advantageously allows to give to the composition of the invention an improved glidance power when applied and an improved homogeneity.

Therefore, the compositions of the invention are more comfortable to wear.

In the invention, a phosphate surfactant useful for preparing a composition of the invention is advantageously free from organogelling functions.

A phosphate surfactant convening to the invention is advantageously also free from plastifying function.

The term “phosphate surfactant” means a surfactant whose polar part comprises at least one phosphate group. The phosphate surfactant may be chosen from the following compounds and mixtures thereof.

The phosphate surfactant may be of formula (XIV): 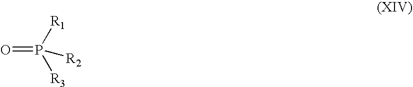
with R₁, R₂ and R₃, which may be identical or different, and which may be chosen from:

a group OM with M representing an alkali metal, such as Na, Li or K, preferably Na or K,

a group OR₄, in which R₄ represents a linear, branched, cyclic or aromatic C₅-C₄₀ alkyl group,

an OH group, and

an oxyethylene group (OCH₂CH₂)ₙ(OCH₂CHCH₃)ₘOR with R representing a hydrogen atom or a linear or branched C₁-C₂₀, for example a C₅-C₁₈ or a C₁₂-C₁₅ alkyl group, and n and m being integers with n ranging from 1 to 50, or being equal to 10, and m ranging from 0 to 50, or being equal to 0.

In one embodiment, R₁, R₂ and R₃ may be identical.

According to another embodiment, at least one, or two, or even all of the radicals R₁, R₂ and R₃ contain(s) a group OR₄, in which R₄ represents a linear or branched C₁₀-C₃₀, in particular C₁₅-C₂₁ or even C₁₈ alkyl group. R₁, R₂ and R₃ may be identical or different.

As non-limiting illustrations of these compounds of formula (XIV), mention may be made for example of trioleyl phosphate such as Nikkol TDP sold by the company Nikko Chemicals, the mixture of triesters of phosphoric acid and of an ether of ethylene glycol and of C₁₂-C₁₅ fatty alcohols (about 10 EO) (INCI name: Tri-C₁₂₋₁₅ Pareth-10 phosphate) such as Nikkol TDP-10 sold by the company Nikko Chemicals, and potassium cetyl phosphate such as Amphisol K sold by the company DSM Nutritional Products or Arlatone MAP 160 K from Uniqema.

The composition may also comprise as phosphate surfactant at least one glycerophospholipid, where appropriate as a mixture with a compound of formula (XIV).

For the purposes of the present invention, the term “glycerophospholipid” is intended to denote an ester obtained by reacting glycerol with at least one saturated or unsaturated fatty acid and phosphoric acid, the said phosphoric acid being substituted with a compound chosen from alcohols bearing an amine function, especially a β-amino alcohol. The β-amino alcohol may be chosen, for example, from choline, ethanolamine and/or serine.

The glycerophospholipid may be defined according to the general formula (XV) below: 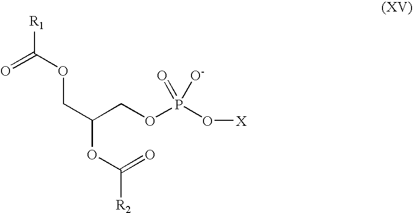
in which:

R₁ and R₂ represent, independently of each other, a saturated or unsaturated, optionally branched fatty acid containing from 4 to 24 carbon atoms, and possibly substituted with one or more hydroxyl and/or amine functions, and

X represents a substituent of general formula R₃R₄R₅N⁺—CH(R₆)—CH₂— in which R₃, R₄, R₅ and R₆ represent, independently of each other, a hydrogen atom, alkyl groups containing from 1 to 6 carbon atoms, and/or a carboxyl function. X may especially be chosen from choline, serine and ethanolamine.

According to one embodiment, R₁ and R₂, independently of each other, are advantageously chosen from butyric acid, caproic acid, caprylic acid, capric acid, caproleic acid, lauric acid, lauroleic acid, myristic acid, myristoleic acid, palmitic acid, palmitoleic acid, stearic acid, oleic acid, linoleic acid, linolenic acid, arachidic acid, isostearic acid, dihydroxystearic acid and ricinoleic acid.

The glycerophospholipid may also be a mixture of compounds of general formula (XV).

The glycerophospholipid that is suitable for use in the invention may for example comprise phosphatidylcholine, phosphatidylethanolamine and/or phosphatidylserine.

According to one embodiment, the glycerophospholipid may comprise an ester of glycerol, of unsaturated fatty acid, of phosphoric acid and of choline, also known as phosphatidylcholine (PC).

The glycerophospholipid that is suitable for use in the invention may be derived from lecithin. The lecithin may comprise, predominantly, phosphatidylcholine as glycerophospholipid.

The phosphatidylcholine (PC) that is suitable for use in the compositions in accordance with the invention may be of “natural” or “synthetic” origin.

“Natural” PC may be obtained by extraction from animal or plant sources, for instance soybean, sunflower or egg. The non-hydrogenated phosphatidylcholine obtained naturally, for instance from soybean, generally contains as glycerol-esterifying fatty acid palmitic acid, stearic acid, palmitoleic acid, oleic acid, linoleic acid, linolenic acid and, optionally, C₂₀-C₂₂ fatty acids.

For the purposes of the present invention, the term “synthetic phosphatidylcholine” is intended to denote phosphatidylcholine comprising at least one fatty acid different from those that may be present in natural PCs.

The term “synthetic PC” is also intended to denote natural PC subjected to modifications, such as partial hydrogenation, i.e. only a fraction of the double bonds present in the unsaturated fatty acids is maintained.

Among the sources of more or less purified phosphatidylcholine that are suitable for use in the cosmetic compositions in accordance with the present invention, mention may be made of Emulmetik 930 sold by the company Lucas Meyer.

The glycerophospholipid that is suitable for use in the present invention may be introduced into the composition in the form of a lecithin. This lecithin is generally obtained by lipid extraction using apolar solvents, from plant or animal fats. This lipid fraction usually comprises, predominantly, glycerophospholipids including phosphatidylcholine or phosphatidylethanolamine.

The lecithins that are suitable for use in the present invention may be lecithins derived from soybean, from sunflower or from egg and/or mixtures thereof.

Lecithins are usually provided in dissolved form in fatty acids, triglycerides or other solvents, or in the form of powders or cakes.

They are usually mixtures of lecithins, in which the glycerophospholipid content in the products as sold generally ranges from about at least 15% to about at least 95%.

In an exemplary embodiment, the lecithin used as starting material for the preparation of the composition according to the invention comprises at least 45% by weight, for example at least 65% by weight, for example at least 75% by weight, for example at least 85% by weight, or for example at least 95% by weight of glycerophospholipid relative to the total weight of the lecithin.

Among the lecithins that may be suitable for use in the cosmetic compositions in accordance with the present invention, mention may be made of the lecithins sold under the references Nattermann Phospholipid®, Phospholipon 80® and Phosale 75® by the company American Lecithin Company, and Epikuron 145V, Topcithin 300, Emulmetik 930 and Ovothin 200 sold by the company Lucas Meyer.

The glycerophospholipid may be a non-hydrogenated glycerophospholipid, i.e. an ester obtained by reacting glycerol with at least one unsaturated fatty acid and phosphoric acid, the said phosphoric acid being substituted with a compound chosen from alcohols bearing an amine function, especially a β-amino alcohol.

The terms “unsaturated” and “unsaturation” are intended to denote the presence of at least one, or even several, double or triple bonds between two carbon atoms.

The glycerophospholipid may be phosphatidylcholine or lecithin.

The phosphate surfactant may be chosen from trioleyl phosphate, the mixture of triesters of phosphoric acid and of an ether of ethylene glycol and of C₁₂-C₁₅ fatty alcohols (about 10 EO), potassium cetyl phosphate, cetyl phosphate and lecithin, and mixtures thereof.

The phosphate surfactant may be a nonionic surfactant such as trioleyl phosphate or the mixture of triesters of phosphoric acid and of an ether of ethylene glycol and of C₁₂-C₁₅ fatty alcohols (about 10 EO).

The phosphate surfactant may be present in the composition according to the invention in a content of between 0.1% and 30%, for example between 0.1% and 10% or for example between 0.5% and 5% by weight relative to the total weight of the composition.

According to a first variant, the composition according to the invention may combine at least trioleyl phosphate and a polyamide/polydimethylsiloxane.

According to a second variant, the composition according to the invention combines at least trioleyl phosphate and a silicone polymer, comprising at least one unit of formula (III) as defined above.

According to a third variant, the composition according to the invention combines at least lecithin and a polyamide/polydimethylsiloxane.

According to a fourth variant, the composition according to the invention combines at least lecithin and a silicone polymer, comprising at least one unit of formula (III) as defined above.

Active Agents

The composition according to the invention may also comprise at least one active agent. The term “active agent” means a compound that has a cosmetic and/or dermatological effect on the lips via a physiological mechanism.

This active agent may be hydrophilic or hydrophobic. The active agent may be water-soluble.

Thus, the active agent present in the composition according to the invention may be chosen independently from:

dermo-relaxing agents,

agents for stimulating the synthesis of dermal or epidermal macromolecules and/or for preventing their degradation,

anti-glycation agents,

anti-irritants,

moisturizers,

desquamating agents,

pigmentation modifiers,

NO-synthase inhibitors,

agents for stimulating fibroblast or keratinocyte proliferation and/or keratinocyte differentiation,

anti-pollution agents or free-radical scavengers,

soothing agents,

agents acting on the capillary circulation,

agents acting on the energy metabolism of cells,

cicatrizing agents, and

mixtures thereof.

Dermo-Relaxing Agents

The composition according to the invention may comprise a hydrophilic active agent and/or a lipophilic active agent chosen from dermo-relaxing agents.

The dermo-relaxing agents that may be used in the composition according to the invention especially comprise alverine and salts thereof, magnesium or manganese chlorides or gluconates, Diazepam, the hexapeptide Argireline R sold by the company Lipotec, the secondary and tertiary carbonyl amines as described in patent application EP-A-1 405 633 (especially 3-[ethyl(3-hydroxy-3-pentyloctyl)amino]propiophenone), adenosine, and also sapogenins and natural extracts, in particular of wild yam, containing them, and also 3-O-acetyl-11-keto boswellic acid and plant extracts containing it such as the extract of Boswellia serrata as described in patent application EP-A-1 442 736; and mixtures thereof.

For example, the dermo-relaxing agents are chosen from magnesium gluconate, manganese gluconate, sapogenins extracted from wild yam, and the extract of Boswellia serrata, and mixtures thereof.

Agents for Stimulating the Synthesis of Dermal or Epidermal Macromolecules and/or for Preventing Their Degradation

The composition according to the invention may comprise a hydrophilic active agent and/or a lipophilic active agent for stimulating the synthesis of dermal or epidermal macromolecules and/or for preventing their degradation.

For example, the agents for stimulating the synthesis of dermal or epidermal macromolecules and/or for preventing their degradation are chosen from extracts of Centella asiatica, ascorbic acid and derivatives thereof, peptides extracted from plants, such as the soybean hydrolysate sold by the company Coletica under the trade name Phytokine®, the extract of Saccharomyces cerevisiae sold by the company LSN under the trade name Cytovitin®; the extract of the brown alga Padina pavonica sold by the company Alban Müller under the trade name HSP3®; retinoids and derivatives; rosemary extracts; the peptide extract of legume seeds (Pisum sativum) sold by the company LSN under the trade name Parelastyl®; {2-[acetyl(3-trifluoromethylphenyl)amino]-3-methylbutyrylamino}acetic acid; extract of lupin; and mixtures thereof.

Anti-Glycation Agents

The composition according to the invention may comprise a hydrophilic active agent or a lipophilic active agent chosen from anti-glycation agents.

The term “anti-glycation agent” means a compound that prevents and/or reduces the glycation of skin proteins, in particular of dermal proteins such as collagen.

Examples of anti-glycation agents are extracts of plants from the Ericacea family, such as an extract of blueberry (Vaccinium angustifolium), for example the product sold under the name Blueberry Herbasol Extract PG by the company Cosmetochem; ergothioneine and derivatives thereof; and hydroxystilbenes and derivatives thereof, such as resveratrol and 3,3′,5,5′-tetrahydroxystilbene. These anti-glycation agents are described in patent applications FR 2 802 425, FR 2 810 548, FR 2 796 278 and FR 2 802 420, respectively. Mention may also be made of arginine and lysine polypeptides such as the product sold under the name Amadorine by the company Solabia; and mixtures thereof.

Blueberry extract may be used as anti-glycation agent.

The composition according to the invention comprising an anti-glycation agent as defined above may be used for preventing or treating the glycation-related signs of ageing of the skin, for example for preventing or treating the age-related loss of tonicity of the lips and the contour of the lips.

Anti-Irritants

The composition according to the invention may comprise a hydrophilic active agent or a lipophilic active agent chosen from anti-irritants.

The term “anti-irritant” means an active agent that modulates the signs of irritation of the lips or of the contour of the lips, such as stinging, tautness and the sensation of heat.

For example, anti-irritants used according to the invention include:

sodium channel blockers, and/or

agents that interact specifically with the receptors of neuromediators or of neurohormones, chosen from substance P antagonists, CGRP antagonists and bradykinin antagonists.

Examples of sodium channel inhibitors that may be used in the invention are: Amiloride, Quinidine, Quinidine sulfate, Apamine, Cyproheptadine, Loperamide and N-acetylprocainamide.

Examples of substance P antagonists that may be used in the invention are especially: strontium salts; spring waters and for example spring water from the Vichy Basin and the spring water from La Roche Posay; bacterial extracts and for example the extract of non-photosynthetic filamentous bacteria described in patent application EP-0 761 204, for example prepared from bacteria belonging to the order of Beggiatoales, or for example to the genera Beggiatoa, Vitreoscilla, Flexithrix or Leucothrix. A strain of Vitreoscilla filiformis may be used according to the invention.

A non-limiting example of a CGRP antagonist that may be used in the present invention consists of an extract of cells (preferably undifferentiated) of at least one plant of the Iridacea family, obtained by in vitro culturing. The Iridacea plant may belong to one of the following genera: Romulea, Crocus, Iris, Gladiolus, Sisyrinchium and Hermodactylus. For use in the present invention, it is possible to use an extract of plant material derived from Iris, for example from Iris pallida, as described in patent application EP-0 765 668.

A non-limiting example of a bradykinin antagonist that may be used in the present invention consists of an extract of at least one plant from the Rosacea family, preferably cultured in vivo. The Rosacea plant extract may belong to the following genera: Agrimonia, Amygdalus, Armeniaca, Cerasus, Malus, Mespilus, Persica, Pirus, Prunus, Rosa, Rubus.

A plant belonging to the genus Rosa may be used according to the invention, for example prepared from material derived from at least one plant belonging to a species chosen from Rosa alba, Rosa alpina, Rosa canina, Rosa cinnamonea, Rosa gallica, Rosa repens, Rosa rubrifolia, Rosa rubiginosa, Rosa sempervirens, Rosa spinosissima, Rosa stylosa, Rosa tomentosa and Rosa villosa. Better still, the plant belongs to the species Rosa gallica as described in patent application EP-0 909 556.

The anti-irritant used according to the invention may be of natural or synthetic origin. The term “natural origin” means an inhibitor in pure form or as a solution, irrespective of its concentration in the said solution, obtained via various processes from a natural component. The term “synthetic origin” means an anti-irritant in pure form or as a solution, irrespective of its concentration in the said solution, obtained via chemical synthesis.

The anti-irritant may also be chosen from Dead Sea salts, extracts of peony, especially of the root of Paeonia suffruticosa, for example sold under the name Botanpi Liquid B by the company Ichimara Pharcos, Dermocalmine from Silab or linseed extracts, such as the product sold under the name Sensiline by the company Silab.

Desquamating Agents

The composition according to the invention may contain a hydrophilic active agent or a lipophilic active agent chosen from desquamating agents.

The term “desquamating agent” means any compound capable of acting:

either directly on desquamation by promoting exfoliation, such as β-hydroxy acids, for example salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives;

or on the enzymes involved in the desquamation or degradation of corneodesmosomes, glycosidases, stratum comeum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; aminosulfonic compounds and for example (N-2-hydroxyethylpiperazine-N-2-ethane)sulfonic acid (HEPES); 2-oxothiazolidine-4-carboxylic acid (procysteine) derivatives; α-amino acid derivatives of the glycine type (such as described in EP-0 852 949 and sodium methylglycinediacetate sold by BASF under the trade name Trilon M); honey; sugar derivatives such as O-octanoyl-6-D-maltose and N-acetylglucosamine.

Moisturizers

The composition according to the invention may contain a hydrophilic active agent or a lipophilic active agent chosen from moisturizers.

The term “moisturizer” means:

either a compound acting on the barrier function, in order to keep the stratum corneum moisturized, or an occlusive compound. Mention may be made of ceramides, sphingoid-based compounds, cholesterol and its derivatives, phytosterols (stigmasterol, β-sitosterol or campesterol), essential fatty acids, 1,2-diacylglycerol, 4-chromanone, pentacyclic triterpenes such as ursolic acid, petroleum jelly and lanolin;

or a compound that directly increases the water content of the stratum corneum, such as threalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides such as the product sold under the reference Pentavitin, honey, alginates (in particular the product Sobalg PH 154 sold by Grindsted), cyclic carbonates, N-lauroylpyrrolidonecarboxylic acid or salts thereof, in particular the sodium salt sold under the reference Nalidone, and N-α-benzoyl-L-arginine;

or a compound that activates the sebaceous glands, such as steroid derivatives (including DHEA, the 7-oxido and/or 17-alkyl derivatives thereof, and sapogenins), methyl dihydrojasmonate and vitamin D and its derivatives.

Depigmenting Anti-Pigmenting or Pro-Pigmenting Agent

The composition according to the invention may contain a hydrophilic active agent or a lipophilic active agent chosen from depigmenting, anti-pigmenting and pro-pigmenting agents.

The depigmenting or anti-pigmenting agents that may be incorporated into the composition according to the present invention comprise, for example, the following compounds: kojic acid; ellagic acid; arbutin and its derivatives such as those described in patent applications EP-895 779 and EP-524 109; hydroquinone; aminophenol derivatives such as those described in patent applications WO 99/10318 and WO 99/32077, and in particular N-cholesteryloxycarbonyl-para-aminophenol and N-ethyloxycarbonyl-para-aminophenol; iminophenol derivatives, for example those described in patent application WO 99/22707; L-2-oxothiazolidine-4-carboxylic acid or procysteine, and also its salts and esters; calcium D-pantheteine sulfonate; ascorbic acid and its derivatives, for example ascorbyl glucoside; and plant extracts, for example extracts of liquorice, of mulberry, of skullcap and of Bacopa monieri, without this list being limiting.

Pro-pigmenting agents that may be mentioned include the extract of burnet (Sanguisorba officinalis) sold by the company Maruzen, and extracts of chrysanthemum (Chrysanthemum morifolium).

NO-Synthase Inhibitors

Examples of NO-synthase inhibitors that are suitable for use in the present invention may comprise a plant extract of the species Vitis vinifera which is sold for example by the company Euromed under the name “Leucocyanidines de raisins extra”, or by the company Indena under the name Leucoselect®, or finally by the company Hansen under the name “Extrait de marc de raisin”; a plant extract of the species Olea europaea which may be obtained from olive tree leaves and is sold for example by the company Vinyals in the form of a dry extract, or by the company Biologia & Technologia under the trade name Eurol BT; and a plant extract of the species Gingko biloba which may be a dry aqueous extract of this plant sold by the company Beaufour under the trade name “Ginkgo biloba extrait standard”.

Agent for Stimulating Fibroblast or Keratinocvte Proliferation and/or Keratinocyte Differentiation

The hydrophilic or lipophilic agents for stimulating fibroblast proliferation that may be used in the composition according to the invention may be chosen, for example, from plant proteins or polypeptides, extracts, for example of soybean (for example an extract of soybean sold by the company LSN under the name Eleseryl SH-VEG 8® or sold by the company Silab under the trade name Raffermine®); and plant hormones such as giberrellins and cytokinins.

The agents for stimulating keratinocyte proliferation that may be used in the composition according to the invention for example comprise retinoids such as retinol and its esters, including retinyl palmitate; adenosine; phloroglucinol; extracts of nut cakes sold by the company Gattefosse; and extracts of Solanum tuberosum sold by the company Sederma.

The agents for stimulating keratinocyte differentiation comprise, for example, minerals such as calcium; the extract of lupin sold by the company Silab under the trade name Photopreventine®; sodium beta-sitosteryl sulfate sold by the company Seporga under the trade name Phytocohesine®; and the extract of corn sold by the company Solabia under the trade name Phytovityl®; and lignans such as secoisolariciresinol.

Anti-Pollution Agents or Free-Radical Scavengers

The composition according to the present invention may contain a hydrophilic active agent or a lipophilic active agent chosen from anti-pollution agents.

The term “anti-pollution agent” means any compound capable of trapping ozone, monocyclic or polycyclic aromatic compounds such as benzopyrene and/or heavy metals such as cobalt, mercury, cadmium and/or nickel. The term “free-radical scavenger” means any compound capable of trapping free radicals.

As ozone-trapping agents that may be used in the composition according to the invention, mention may be made for example of vitamin C and its derivatives including ascorbyl glucoside; phenols and polyphenols, for example tannins, ellagic acid and tannic acid; epigallocatechin and natural extracts containing it; extracts of olive tree leaf; extracts of tea, for example of green tea; anthocyans; extracts of rosemary; phenolic acids, in particular chlorogenic acid; stilbenes, in particular resveratrol; sulfur-containing amino acid derivatives, in particular S-carboxymethylcysteine; ergothioneine; N-acetylcysteine; chelating agents, for instance N,N′-bis(3,4,5-trimethoxybenzyl)ethylenediamine or one of its salts, metal complexes or esters; carotenoids such as crocetin; and various starting materials, for instance the mixture of arginine, histidine ribonucleate, mannitol, adenosine triphosphate, pyridoxine, phenylalanine, tyrosine and hydrolysed RNA, sold by Laboratoires Sérobiologiques under the trade name CPP LS 2633-12F®, the water-soluble fraction of corn sold by the company Solabia under the trade name Phytovityl®, the mixture of extract of fumitory and of extract of lemon sold under the name Unicotrozon C-49® by the company Induchem, and the mixture of extracts of ginseng, of apple, of peach, of wheat and of barley, sold by the company Provital under the trade name Pronalen Bioprotect®.

As agents for trapping monocyclic or polycyclic aromatic compounds, which may be used in the composition according to the invention, mention may be made of tannins such as ellagic acid; indole derivatives, in particular 3-indolecarbinol; extracts of tea, in particular of green tea, extracts of water hyacinth or Eichhornia crassipes; and the water-soluble fraction of corn sold by the company Solabia under the trade name Phytovityl®.

Finally, as heavy-metal-trapping agents that may be used in the composition according to the invention, mention may be made of chelating agents such as EDTA, the pentasodium salt of ethylenediaminetetramethylenephosphonic acid, and N,N′-bis(3,4,5-trimethoxybenzyl)ethylenediamine or one of the salts, metal complexes or esters thereof; phytic acid; chitosan derivatives; extracts of tea, in particular of green tea; tannins such as ellagic acid; sulfur-containing amino acids such as cysteine; extracts of water hyacinth (Eichhornia crassipes); and the water-soluble fraction of corn sold by the company Solabia under the trade name Phytovityl®.

The free-radical scavengers that may be used in the composition according to the invention comprise, besides certain anti-pollution agents mentioned above, vitamin E and its derivatives such as tocopheryl acetate; bioflavonoids; coenzyme Q10 or ubiquinone; certain enzymes, for instance catalase, superoxide dismutase and extracts of wheatgerm containing it, lactoperoxidase, glutathione peroxidase and quinone reductases; glutathione; benzylidenecamphor; benzylcyclanones; substituted naphthalenones; pidolates; phytanetriol; gamma-oryzanol; guanosine; lignans; and melatonin.

Soothing Agents

As soothing agents that may be used in the composition according to the invention, mention may be made of: pentacyclic triterpenes and extracts of plants (e.g.: Glycyrrhiza glabra) containing them, for instance β-glycyrrhetinic acid and salts and/or derivatives thereof (glycyrrhetinic acid monoglucuronide, stearyl glycyrrhetinate or 3-stearoyloxyglycyrrhetic acid), ursolic acid and its salts, oleanolic acid and its salts, betulinic acid and its salts, extracts of plants such as Paeonia suffruticosa and/or lactiflora, Laminaria saccharina, Boswellia serrata, Centipeda cunninghami, Helianthus annuus, Linum usitatissimum, Cola nitida, Epilobium angustifolium, Aloe vera, Bacopa monieri, salicylic acid salts and in particular zinc salicylate, canola oil, bisabolol and camomile extracts, allantoin, Sepivital EPC (phosphoric diester of vitamins E and C) from SEPPIC, omega-3 unsaturated oils such as musk rose oil, blackcurrant oil, ecchium oil, fish oil, plankton extracts, capryloylglycine, Seppicalm VG (sodium palmitoylproline and Nymphea alba) from SEPPIC, tocotrienols, piperonal, an extract of clove, phytosterols, cortisone, hydrocortisone, indomethacin and betamethasone.

Agents Acting on the Capillary Circulation

The active agents acting on the capillary circulation (vasoprotective or vasodilating agents) may be chosen from flavonoids, ruscogenins, esculosides, escin extracted from common horsechestnut, nicotinates, heperidine methyl chalcone, essential oils of lavender or of rosemary, and extracts of Ammi visnaga.

Agents Acting on the Energy Metabolism of Cells

The active agents concerned are those acting on the energy metabolism of the skin, for instance, and in a non-limiting manner, ATP synthesis, and also those involved in the respiratory chain of the cell or in the energy reserves. Mention may be made of coenzyme Q10 (ubiquinone), cytochrome C, creatine or phosphocreatine.

Cicatrizing Agents

Examples of cicatrizing agents are especially the extract of fern leaves sold under the reference Mamaku Vital Essence by Lucas Meyer, and rice peptides obtained by hydrolysing rice protein, sold under the name Nutripeptide by Silab.

Among the hydrophilic active agents and lipophilic active agents that may be included in the composition according to the invention, mention may also be made of active agents chosen from carotenoids (α-carotene, β-carotene; zeaxanthin; lutein; ataxanthin, bixin), canthaxanthin, cryptoxanthin, retinoids (retinol, retinal, 13-cis-retinoic acid, all-trans-retinoic acid), flavanones (naringenin and hesperetin, glycosyl hesperetin), flavonols (kaempferol, quercetol), isoflavones (isoflavone, daidzein, genistein), coumarins (esculoside, visnadine, esculetol, 4-methylesculetol, Permethol from Syphetal such as the sodium salt of methylesculetin acetate), lignans (silymarin, nordihydroguaiaretic acid, secoisolariciresinol), stilbenoids (resveratrol and the alkoxyl and glycosyl derivatives thereof), sapogenins (hederagenin, diosgenin, hecogenin and smilagenin), pentacyclic triterpenic acids (glycyrrhetinic acid, glycyrrhizic acid, ursolic acid, betulinic acid, oleanic acid, asiatic acid, madecassic acid), sterols (such as cholesterol or fucosterol), hydroxyphenols and derivatives thereof (hydroquinone, arbutin, homogentisic acid, gentisic acid, catechol, guaiacol, resorcinol, lucinol, mequinol), phenolic acids (cinnamic acid, coumarinic acid, caffeic acid, rosmarinic acid, ferulic acid, chlorogenic acid), tannin-precursor monomers (gallic acid, ellagic acid, extracts of hamamelis), amino sugars (N-acetylglucosamine, N-acetylgalactosamine), vitamins chosen from vitamin A, vitamin C, vitamin E, vitamin B3, vitamin B5,vitamin D, vitamin F, and derivatives, analogues and precursors thereof, and mixtures thereof.

The amount of active agent(s) ranges, for example, from 0.001% to 30% by weight, for example from 0.01% to 20% by weight of active material relative to the total weight of the composition.

Physiologically Acceptable Medium

The composition of the invention should be cosmetically or dermatologically acceptable, i.e. it should contain a non-toxic physiologically acceptable medium that may be applied to human lips. For the purposes of the invention, the term “cosmetically acceptable” means a composition of pleasant appearance, odour and feel.

Fatty Phase

The physiologically acceptable medium may comprise a fatty phase.

The fatty phase of the composition according to the invention may be a liquid fatty phase based on at least one oil.

Liquid Fatty Phase

The oil may be a silicone oil, an ester oil or a non-silicone oil.

a. Silicone Oil

According to one variant of the invention, the liquid fatty phase comprises at least one volatile silicone oil.

For the purposes of the invention, a volatile oil has at room temperature (25° C.) and atmospheric pressure (760 mm Hg) a vapour pressure ranging from 0.02 mm Hg to 300 mm Hg (2.66 Pa to 40 000 Pa), for example ranging from 0.1 mm Hg to 90 mm Hg (13 Pa to 12 000 Pa). The non-volatile oils then correspond to a vapour pressure of less than 0.02 mm Hg (2.66 Pa).

The volatile silicone oil may be chosen from linear or cyclic silicone oils, such as linear or cyclic polydimethylsiloxanes (PDMS) containing from 3 to 7 silicon atoms.

Examples of such oils that may be mentioned include octyl trimethicone, hexyl trimethicone, decamethylcyclopentasiloxane (cyclopentasiloxane or D5), octamethylcyclotetrasiloxane, (cyclotetradimethylsiloxane or D4), dodecamethylcyclohexasiloxane (D6), decamethyltetrasiloxane (L4), KF 96 A from Shin-Etsu, and polydimethylsiloxanes such as those sold under the references DC 200 (1.5 cSt), DC 200 (5 cSt) and DC 200 (3 cSt) from Dow Corning.

The non-volatile silicone oils may be polydimethylsiloxanes, polyalkylmethylsiloxanes, dimethicone copolyols, alkylmethicone copolyols, cetyl dimethicone, silicones containing alkylglyceryl ether groups, silicones containing amine side groups and dilauroyltrimethylolpropane siloxysilicate. The alkyl groups of these oils may contain from 2 to 24 carbon atoms.

The non-volatile silicone oils that may be used in the liquid fatty phase may be linear non-volatile polydimethylsiloxanes (PDMS) that are liquid at room temperature; polydimethylsiloxanes comprising alkyl, alkoxy or phenyl groups, which are pendent and/or at the end of a silicone chain, these groups each containing from 2 to 24 carbon atoms; phenyl silicones, for instance phenyl trimethicones, phenyl dimethicones, phenyl trimethylsiloxy diphenylsiloxanes, diphenyl dimethicones, diphenyl methyldiphenyl trisiloxanes, 2-phenylethyl trimethylsiloxysilicates, fluoro silicones with groups that are pendent or at the end of a chain, containing from 1 to 12 carbon atoms, all or some of the hydrogen atoms of which are substituted with fluorine atoms, dimethiconols, and mixtures thereof.

The silicone oils have a viscosity advantageously chosen in the range from 5 to 800 000 cSt, for example from 10 to 500 000 cSt, or for example from 10 to 5000 cSt at 25° C.

The liquid fatty phase advantageously may contain from 0.1% to 60%, for example from 5% to 50%, by weight of silicone oil(s).

b. Ester Oil

According to one variant of the invention, at least one of the oils of the liquid fatty phase is an oil known as an “ester oil”, which is chosen from esters of monocarboxylic acids with monoalcohols and polyalcohols.

In one embodiment, the said ester corresponds to the following formula:
R₁—CO—O—R₂
where R₁ represents a linear or branched alkyl radical of 1 to 40 carbon atoms, for example of 7 to 19 carbon atoms, optionally comprising one or more ethylenic double bonds, and optionally substituted.

R₂ represents a linear or branched alkyl radical of 1 to 40 carbon atoms, for example of 3 to 30 carbon atoms, or for example and better still of 3 to 20 carbon atoms, optionally comprising one or more ethylenic double bonds, and optionally substituted.

The term “optionally substituted” means that R₁ and/or R₂ can bear one or more substituents chosen, for example, from groups comprising one or more hetero atoms chosen from O, N and S, such as amino, amine, alkoxy and hydroxyl.

In one exemplary embodiment, the total number of carbon atoms of R₁+R₂ is ≧9.

R₁ may represent the residue of a linear or, for example, branched fatty acid, preferably a higher fatty acid, containing from 1 to 40, for example from 7 to 19 carbon atoms, and R₂ may represent a linear or, for example, branched hydrocarbon-based chain containing from 1 to 40, for example from 3 to 30 and even better from 3 to 20 (19 to 28, 8 to 27, 7 to 26 C) carbon atoms. In one embodiment, the number of carbon atoms of R₁+R₂≧9.

Examples of groups R₁ are those derived from fatty acids chosen from the group consisting of acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, oleostearic acid, arachidonic acid and erucic acid, and mixtures thereof.

Examples of esters that may be used in the fatty phases of the compositions of the invention include purcellin oil (cetostearyl octanoate), isononyl isononanoate, isopropyl myristate, 2-ethylhexyl palmitate, 2-octyldodecyl stearate, 2-octyldodecyl erucate, isostearyl isostearate, and heptanoates, octanoates, decanoates or ricinoleates of alcohols or polyalcohols, for example of fatty alcohols.

In one embodiment, the esters are chosen from the compounds of formula (I) above, in which R₁ represents an unsubstituted linear or branched alkyl group of 1 to 40 carbon atoms, or for example of 7 to 19 carbon atoms, optionally comprising one or more ethylenic double bonds, and R₂ represents an unsubstituted linear or branched alkyl group of 1 to 40 carbon atoms, for example of 3 to 30 carbon atoms, or for example of 3 to 20 carbon atoms, optionally comprising one or more ethylenic double bonds.

In one embodiment, R₁ is an unsubstituted branched alkyl group of 4 to 14 carbon atoms, for example of 8 to 10 carbon atoms, and R₂ is an unsubstituted branched alkyl group of 5 to 15 carbon atoms, or for example of 9 to 11 carbon atoms. For example, in formula (I), R₁—CO— and R₂ have the same number of carbon atoms and are derived from the same radical, for example an unsubstituted branched alkyl, for example isononyl, i.e. the ester oil molecule may be symmetrical.

The ester oil may be chosen from the following compounds:

isononyl isononanoate,

cetostearyl octanoate,

isopropyl myristate,

2-ethylhexyl palmitate,

2-octyldodecyl stearate,

2-octyldodecyl erucate,

isostearyl isostearate.

The ester that may convene among all of them is isononyl isononanoate.

According to one embodiment, the composition comprises less than 10% of volatile ester oil, for example less than 5% of volatile ester oil, or for example till less than 3% of volatile ester oil, or is even free of volatile ester oil.

In one embodiment, the liquid fatty phase comprises from 0.1% to 60% by weight, for example from 5% to 50% by weight of ester oil(s).

c. Non-Silicone Oil

The liquid fatty phase of the compositions according to the invention may also contain one or more volatile or non-volatile non-silicone oils. The volatile non-silicone oils may be chosen from the group of volatile hydrocarbon-based oils, esters and ethers, such as volatile hydrocarbons, for instance isododecane and isohexadecane, and C₈-C₁₆ isoparaffins.

The volatile non-silicone oil may also be chosen from fluoro oils such as perfluoropolyethers, perfluoroalkanes, for instance perfluorodecalin, perfluoroadamantanes, perfluoroalkyl phosphate monoesters, diesters and triesters, and fluoro ester oils.

As examples of volatile non-silicone oils that may be used in the composition of the invention, mention may be made of isododecane, isohexadecane, propylene glycol n-butyl ether, ethyl 3-ethoxypropionate, propylene glycol methyl ether acetate, C₁₁-C₁₃ isoparaffins such as Isopar L® or C₁₁-C₁₂ isoparaffins such as Isopar H®.

When the fatty phase comprises a volatile non-silicone oil, it may represent from 0.1% to 60%, or for example from 5% to 20%, relative to the total weight of the composition.

The liquid fatty phase may also contain other non-silicone oils, for example polar oils such as:

hydrocarbon-based plant oils with a high content of triglycerides consisting of fatty acid esters of glycerol in which the fatty acids may have varied chain lengths, these chains possibly being linear or branched, and saturated or unsaturated; these oils are for example wheatgerm oil, corn oil, sunflower oil, shea oil, castor oil, sweet almond oil, macadamia oil, apricot oil, soybean oil, rapeseed oil, cottonseed oil, alfalfa oil, poppy seed oil, pumpkin seed oil, sesame seed oil, marrow oil, avocado oil, hazelnut oil, grapeseed oil, blackcurrant seed oil, evening primrose oil, millet oil, barley oil, quinoa oil, olive oil, rye oil, safflower oil, candlenut oil, passion flower oil and musk rose oil; or caprylic/capric acid triglycerides such as those sold by the company Stearines Dubois or those sold under the names Miglyol 810, 812 and 818 by the company Dynamit Nobel;

synthetic ethers containing from 10 to 40 carbon atoms;

C₈ to C₂₆ fatty alcohols, for instance oleyl alcohol and octyldodecanol;

fatty acids, for instance oleic acid, linoleic acid or linolenic acid; and

mixtures thereof.

The liquid fatty phase may also contain apolar oils such as linear or branched, volatile or non-volatile hydrocarbons or fluorocarbons of synthetic or mineral origin, for instance volatile liquid paraffins (such as isoparaffins or isododecane) or non-volatile liquid paraffins and derivatives thereof, petroleum jelly, polydecenes, hydrogenated polyisobutene such as Parleam, and squalane, and mixtures thereof.

The oil(s) may be present in the composition according to the invention in a content ranging from 0.1% to 90% by weight, for example from 5% to 80% by weight relative to the total weight of the composition.

Structuring Agent

The composition according to the invention may comprise, besides the silicone polymer(s) described above, a structuring agent chosen from waxes, semi-crystalline polymers and lipophilic gelling agents, and mixtures thereof.

It is understood that the amount of these additional compounds may be adjusted by a person skilled in the art so as not to harm the desired effect in the context of the present invention.

Wax(es)

The wax under consideration in the context of the present invention is generally a lipophilic compound that is solid at room temperature (25° C.), with a solid/liquid reversible change of state, having a melting point of greater than or equal to 30° C., which may be up to 200° C. and in particular up to 120° C.

By bringing the wax to the liquid form (melting), it is possible to make it miscible with oils and to form a microscopically uniform mixture, but on cooling the mixture to room temperature, recrystallization of the wax in the oils of the mixture is obtained.

In on embodiment, the waxes that are suitable for the invention may have a melting point of greater than or equal to 45° C., for example greater than or equal to 55° C.

For the purposes of the invention, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The waxes that may be used in the compositions according to the invention are chosen from waxes that are solid at room temperature of animal, plant, mineral or synthetic origin, and mixtures thereof.

The waxes that may be used in the compositions according to the invention generally have a hardness ranging from 0.01 MPa to 15 MPa, for example greater than 0.05 MPa, or for example greater than 0.1 MPa.

As illustrations of waxes that are suitable for the invention, mention may be made of hydrocarbon-based waxes, for instance beeswax, lanolin wax, Chinese insect waxes, rice bran wax, carnauba wax, candelilla wax, ouricurry wax, esparto grass wax, berry wax, shellac wax, Japan wax and sumach wax; montan wax, orange wax and lemon wax, microcrystalline waxes, paraffins and ozokerite; polyethylene waxes, the waxes obtained by Fischer-Tropsch synthesis and waxy copolymers, and also esters thereof.

Mention may also be made of waxes obtained by catalytic hydrogenation of animal or plant oils containing linear or branched C₈-C₃₂ fatty chains. Among these waxes that may be mentioned are isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane)tetrastearate sold under the name Hest 2T-4S® by the company Heterene.

Mention may also be made of silicone waxes (C₃₀₋₄₅ alkyl dimethicone) and fluoro waxes.

The waxes obtained by hydrogenation of castor oil esterified with cetyl alcohol, sold under the names Phytowax ricin 16L64® and 22L73® by the company Sophim, may also be used. Such waxes are described in patent application FR-A-2 792 190.

A wax that may be used is a C₂₀-C₄₀ alkyl (hydroxystearyloxy)stearate (the alkyl group containing from 20 to 40 carbon atoms), alone or as a mixture.

Such a wax is for example sold under the names Kester Wax K 82 P®, Hydroxypolyester K 82 P® and Kester Wax K 80 P® by the company Koster Keunen.

As microwaxes that may be used in the compositions according to the invention, mention may be made for example of carnauba microwaxes, such as the product sold under the name MicroCare 350® by the company Micro Powders, synthetic microwaxes, such as the product sold under the name MicroEase 114S® by the company Micro Powders, microwaxes consisting of a mixture of carnauba wax and polyethylene wax, such as the products sold under the names Micro Care 300® and 310® by the company Micro Powders, microwaxes consisting of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

The composition according to the invention may comprise a content of waxes ranging from 0. 1% to 30% by weight relative to the total weight of the composition; it may for example contain from 0.5% to 15%, or for example from 1% to 10% thereof.

Pasty Compounds

The composition according to the invention may also contain a pasty compound, which may be chosen from:

lanolin and its derivatives

polymeric or non-polymeric silicone compounds

polymeric or non-polymeric fluoro compounds

vinyl polymers, for example:
olefin homopolymers
olefin copolymers
hydrogenated diene homopolymers and copolymers
linear or branched oligomers, homopolymers or copolymers of alkyl(meth)acrylates, for example containing a C₈-C₃₀ alkyl group
oligomers, homopolymers and copolymers of vinyl esters containing C₈-C₃₀ alkyl groups
oligomers, homopolymers and copolymers of vinyl ethers containing C₈-C₃₀ alkyl groups

liposoluble polyethers resulting from the polyetherification between one or more C₂-C₁₀₀ and preferably C₂-C₅₀ diols

esters, and

mixtures thereof.

Among the esters, the following may be used:

esters of a glycerol oligomer, for example diglycerol esters, such as condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, especially such as those sold under the brand name Softisan 649 by the company Sasol,

the arachidyl propionate sold under the brand name Waxenol 801 by Alzo,

phytosterol esters,

fatty acid triglycerides and derivatives thereof,

pentaerythritol esters,

non-crosslinked polyesters resulting from the polycondensation between a linear or branched C₄-C₅₀ dicarboxylic acid or polycarboxylic acid and a C₂-C₅₀ diol or polyol,

aliphatic esters of an ester, resulting from the esterification of an aliphatic hydroxycarboxylic acid ester with an aliphatic carboxylic acid,

polyesters resulting from the esterification, with a polycarboxylic acid, of an aliphatic hydroxycarboxylic acid ester, the said ester comprising at least two hydroxyl groups, such as the products Risocast DA-H® and Risocast DA-L®, and

mixtures thereof.

Among the pasty compounds of plant origin that may be chosen is a mixture of oxyethylenated (5 OE) oxypropylenated (5 OP) soybean sterols and pentaerythritol, sold under the reference Lanolide by the company Vevy.

According to one embodiment, the composition comprises less than 10% by weight, for example less than 7%, for example less than 5%, or for example less than 3% by weight of wax relative to the total weight of the composition. In one embodiment, the composition is totally free of wax.

Lipophilic Gelling Agents

The gelling agents that may be used in the compositions according to the invention may be organic or mineral, polymeric or molecular lipophilic gelling agents.

Mineral lipophilic gelling agents that may be mentioned include optionally modified clays, for instance hectorites modified with a C₁₀ to C₂₂ fatty acid ammonium chloride, for instance hectorite modified with distearyldimethylammonium chloride, for instance the product sold under the name Bentone 38V® by the company Elementis.

Mention may also be made of fumed silica optionally subjected to a hydrophobic surface treatment, the particle size of which is less than 1 μm. For example, it is possible to chemically modify the surface of the silica, by chemical reaction generating a reduced number of silanol groups present at the surface of the silica. It is for example possible to substitute silanol groups with hydrophobic groups: a hydrophobic silica is then obtained. The hydrophobic groups may be:

trimethylsiloxyl groups, which may be obtained by treating fumed silica in the presence of hexamethyldisilazane. Silicas thus treated are known as Silica Silylate according to the CTFA (6th edition, 1995). They are sold, for example, under the references Aerosil R812® by the company Degussa, and Cab-O-Sil TS-530® by the company Cabot;

dimethylsilyloxyl or polydimethylsiloxane groups, which may be obtained by treating fumed silica in the presence of polydimethylsiloxane or dimethyldichlorosilane. Silicas thus treated are known as Silica Dimethyl Silylate according to the CTFA (6th edition, 1995). They are sold, for example, under the references Aerosil R972® and Aerosil R974® by the company Degussa, and Cab-O-Sil TS-610® and Cab-O-Sil TS-720® by the company Cabot.

The hydrophobic fumed silica may have a particle size that may be nanometric to micrometric, for example ranging from about 5 to 200 nm.

The polymeric organic lipophilic gelling agents are, for example, partially or totally crosslinked elastomeric organopolysiloxanes of three-dimensional structure, for instance those sold under the names KSG6®, KSG16® and KSG18® from Shin-Etsu, Trefil E-505C® or Trefil E-506C® from Dow Corning, Gransil SR-CYC®, SRDMF10®, SR-DC556®, SR SCYC gel®, SR DMF 10 gel® and SR DC 556 gel® from Grant Industries and SF 1204® and JK 113® from General Electric; ethylcellulose, for instance the product sold under the name Ethocel® by Dow Chemical; polycondensates of polyamide type resulting from condensation between (α) at least one acid chosen from dicarboxylic acids containing at least 32 carbon atoms, such as fatty acid dimers, and (β) an alkylenediamine and in particular ethylenediamine, in which the polyamide polymer comprises at least one carboxylic acid end group esterified or amidated with at least one saturated and linear monoalcohol or one saturated and linear monoamine containing from 12 to 30 carbon atoms, for example ethylenediamine/stearyl dilinoleate copolymers such as the product sold under the name Uniclear 100 VG® by the company Arizona Chemical; galactomannans comprising from one to six, or for example from two to four hydroxyl groups per saccharide, substituted with a saturated or unsaturated alkyl chain, for instance guar gum alkylated with C₁ to C₆, for example with C₁ to C₃, alkyl chains, and mixtures thereof. Block copolymers of “diblock”, “triblock” or “radial” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Shell Chemical Co., or of the polystyrene/copoly(ethylene-butylene) type, and mixtures of triblock and radial (star) copolymers in isododecane, such as those sold by the company Penreco under the name Versagel®, for instance the mixture of butylene/ethylene/styrene triblock copolymer and of ethylene/propylene/styrene star copolymer in isododecane (Versagel M 5960).

Among the lipophilic gelling agents that may be used in the compositions according to the invention, mention may also be made of fatty acid esters of dextrin, such as dextrin palmitates, especially the products sold under the name Rheopearl TL® or Rheopearl KL® by the company Chiba Flour.

Aqueous Phase

The physiologically acceptable medium of the composition according to the invention may comprise an aqueous medium, constituting an aqueous phase, which may form the continuous phase of the composition.

The aqueous phase may consist essentially of water; it may also comprise a mixture of water and of water-miscible solvent (miscibility in water of greater than 50% by weight at 25° C.), for instance lower monoalcohols containing from 1 to 5 carbon atoms, such as ethanol or isopropanol, glycols containing from 2 to 8 carbon atoms, such as propylene glycol, ethylene glycol, 1,3-butylene glycol and dipropylene glycol, C₃-C₄ ketones and C₂-C₄ aldehydes, and mixtures thereof.

The aqueous phase (water and optionally the water-miscible solvent) may be present in a content ranging from 1% to 95% by weight, for example ranging from 2% to 80% by weight, or for example ranging from 3% to 60% by weight relative to the total weight of the composition.

The composition according to the invention may also contain less than 10% by weight or for example less than 4% by weight of aqueous phase or of water.

According to one preferred embodiment, the composition is anhydrous.

Emulsifying System

Besides the phosphate surfactant, the compositions according to the invention may also contain one or more surfactants.

According to one variant of the invention, they comprise at least one silicone surfactant.

The silicone surfactant(s) may be present in the composition in a content ranging from 0.1% to 50% by weight, for example ranging from 0.1% to 40% by weight, for example ranging from 0.5% to 30% by weight, for example ranging from 0.5% to 20% by weight, or for example ranging from 1% to 10% by weight, relative to the total weight of the composition.

Among the surfactants that may be used in the cosmetic compositions in accordance with the present invention, mention may be made of hydrophilic organopolysiloxanes other than the silicone polymer described hereinabove.

The hydrophilic radical may correspond to the formula: [symbol]
CH₂ [symbol] ₚO [symbol] C₂H₄O)_{q} [symbol] C₃H₆O [symbol] ᵣX
in which

p ranges from 0 to 5, q ranges from 0 to 100 and r ranges from 0 to 50, with p or q being non-zero,

the units (C₂H₄O) and (C₃H₆O) may be distributed randomly or in blocks, and

X is a hydrogen or a C₁-C₁₀ alkyl radical, where appropriate substituted with one or more functions of hydroxyl, thiol, amine, carboxylic, carboxylate, amide, phosphate, sulfate or sulfonate type.

In one embodiment, p may range from 1 to 5, q from 1 to 100 and r from 1 to 50. X may for example feature a hydrogen atom.

In one embodiment, the organopolysiloxane according to the invention may comprise as hydrophilic radical at least one hydroxy-polyalkylenoxy radical, for example a hydroxy-polyethylenoxy radical.

The organopolysiloxane according to the invention may correspond to the formula: 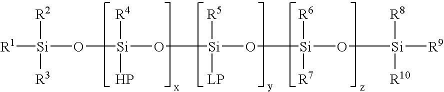
in which:

R¹, R², R³, R⁴, R⁵, R⁶, R⁷, R⁸, R⁹ and R¹⁰ represent, independently of each other, a linear, branched or cyclic, saturated or unsaturated C₁-C₆ alkyl radical,

HP is a radical bearing at least one hydrophilic group as defined hereinabove,

LP is a lipophilic radical, and

x ranges from 1 to 5000; y from 0 to 5000; z from 0 to 5000.

As regards the radical LP, it may be chosen from linear, branched or cyclic C₁-C₄₀ alkyls, organosiloxane groups, fluorine atoms and aryl, aryloxy, C₁-C₄₀ hydrocarbyl acyl and hydroxypropylenoxy radicals.

According to one particular variant of the invention, the organopolysiloxane belongs to the family of dimethicone copolyols, for example of cetyldimethicone copolyol Iand derivatives thereof. The hydrophilic organopolysiloxane according to the present invention may be the product sold under the brand name Abil WE 09 or Abil EM 90 by the company Degussa-Goldschmidt. The hydrophilic organopolysiloxane according to the present invention may also be the product sold under the reference KF-6017 by the company Shin-Etsu.

The organopolysiloxane compound may be totally or partially fluorinated. In an embodiment, the lower dialkyl siloxy groups may be substituted with one or more fluorine atoms.

According to one particular embodiment, the silicone surfactant that may be used in the cosmetic compositions in accordance with the present invention may be chosen from dimethicone copolyol, dimethicone copolyol benzoate, dimethicone copolyol phosphates, polyoxyalkylenated silicone elastomers and the cyclomethicone/dimethicone mixture, and mixtures thereof.

Polyoxyalkylenated silicone elastomers such as those described in patents U.S. Pat. No. 5,236,986, U.S. Pat. No. 5,412,004, U.S. Pat. No. 5,837,793 and U.S. Pat. No. 5,811,487, the content of which is incorporated by reference, are also suitable according to the invention.

Polyoxyalkylenated silicone elastomers that may be used include those sold under the names KSG-21, KSG-20, KSG-30, KSG-31, KSG-32, KSG-33, KSG-210, KSG-310, KSG-320, KSG-330, KSG-340 and X-226146 by the company Shin-Etsu, and DC 9010 and DC 9011 by the company Dow Corning.

It is understood that the compositions according to the invention may also comprise anionic and/or nonionic non-silicone surfactants.

For the choice of these surfactants, reference may be made to Kirk-Othmer's “Encyclopedia of Chemical Technology”, volume 22, pp. 333-432, 3rd edition, 1979, Wiley, for the definition of the properties and functions (emulsifying) of surfactants, in particular pp. 347-377 of this reference, for the anionic and nonionic surfactants.

The surfactants that may be used more particularly in the composition according to the invention may be chosen from:

nonionic surfactants: fatty acids, fatty alcohols, polyethoxylated or polyglycerolated fatty alcohols such as polyethoxylated stearyl or cetylstearyl alcohol, fatty acid esters of sucrose, alkyl glucose esters, for example polyoxyethylenated fatty esters of a C₁-C₆ alkyl glucose, and mixtures thereof,

anionic surfactants: C₁₆-C₃₀ fatty acids neutralized with amines, ammonia or alkaline salts, and mixtures thereof.

Film-Forming Polymer

According to one particular embodiment, the composition according to the invention may comprise at least one film-forming polymer.

The film-forming polymer may be present in the composition according to the invention in a solids (or active material) content ranging from 0.1% to 30% by weight, for example from 0.5% to 20% by weight, or for example from 1% to 15% by weight relative to the total weight of the composition.

In the present invention, the expression “film-forming polymer” means a polymer that is capable, by itself or in the presence of an auxiliary film-forming agent, of forming a macroscopically continuous film, for example when the said film is made by casting on a non-stick surface, for instance a Teflon-coated or silicone-coated surface.

Among the film-forming polymers that may be used in the composition of the present invention, mention may be made of synthetic polymers, of free-radical type or of polycondensate type, and polymers of natural origin, and mixtures thereof.

The expression “free-radical film-forming polymer” means a polymer obtained by polymerization of unsaturated, for example ethylenically unsaturated monomers, each monomer being capable of homopolymerizing (unlike polycondensates).

The film-forming polymers of free-radical type may be, for example, vinyl polymers or copolymers, for example acrylic polymers.

The vinyl film-forming polymers can result from the polymerization of ethylenically unsaturated monomers containing at least one acidic group and/or esters of these acidic monomers and/or amides of these acidic monomers.

Monomers bearing an acidic group that may be used are α,β-ethylenic unsaturated carboxylic acids such as acrylic acid, methacrylic acid, crotonic acid, maleic acid or itaconic acid. (Meth)acrylic acid and crotonic acid are preferably used, and more preferably (meth)acrylic acid.

The esters of acidic monomers may be chosen from (meth)acrylic acid esters (also known as (meth)acrylates), especially (meth)acrylates of an alkyl, for example of a C₁-C₃₀ and preferably C₁-C₂₀ alkyl, (meth)acrylates of an aryl, for example of a C₆-C₁₀ aryl, and (meth)acrylates of a hydroxyalkyl, for example of a C₂-C₆ hydroxyalkyl.

Among the alkyl(meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl(meth)acrylates that may be mentioned are hydroxyethyl acrylate, 2-hydroxypropyl acrylate, hydroxyethyl methacrylate and 2-hydroxypropyl methacrylate.

Among the aryl(meth)acrylates that may be mentioned are benzyl acrylate and phenyl acrylate.

The (meth)acrylic acid esters that may be used are the alkyl(meth)acrylates.

According to the present invention, the alkyl group of the esters may be either fluorinated or perfluorinated, i.e. some or all of the hydrogen atoms of the alkyl group are substituted with fluorine atoms.

Examples of amides of the acid monomers that may be mentioned are (meth)acrylamides, and especially N-alkyl(meth)acrylamides, in particular of a C₂-C₁₂ alkyl. Among the N-alkyl(meth)acrylamides that may be mentioned are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with acid monomers and/or esters thereof and/or amides thereof, such as those mentioned above.

Examples of vinyl esters that may be mentioned are vinyl acetate, vinyl neodecanoate, vinyl pivalate, vinyl benzoate and vinyl t-butylbenzoate.

Styrene monomers that may be mentioned are styrene and α-methylstyrene.

Among the film-forming polycondensates that may be mentioned are polyurethanes, polyesters, polyesteramides, polyamides, epoxyester resins and polyureas.

The polyurethanes may be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurealpolyurethanes, and mixtures thereof.

The polyesters may be obtained, in a known manner, by polycondensation of dicarboxylic acids with polyols, in particular diols.

The dicarboxylic acid may be aliphatic, alicyclic or aromatic. Examples of such acids that may be mentioned are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers may be used alone or as a combination of at least two dicarboxylic acid monomers. Among these monomers, the ones preferentially chosen are phthalic acid, isophthalic acid and terephthalic acid.

The diol may be chosen from aliphatic, alicyclic and aromatic diols. The diol used may be chosen from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

The polyester may also comprise at least one monomer bearing at least one group —SO₃M, with M representing a hydrogen atom, an ammonium ion NH4⁺or a metal ion such as, for example, an Na⁺, Li⁺, K⁺, Mg²⁺, Ca²⁺, Cu²⁺, Fe²⁺ or Fe³⁺ ion. A difunctional aromatic monomer comprising such a group —SO₃M may be used in particular.

The aromatic nucleus of the difunctional aromatic monomer also bearing a group —SO₃M as described above may be chosen, for example, from benzene, naphthalene, anthracene, biphenyl, oxybiphenyl, sulfonylbiphenyl and methylenebiphenyl nuclei. As examples of difunctional aromatic monomers also bearing a group —SO₃M, mention may be made of: sulfoisophthalic acid, sulfoterephthalic acid, sulfophthalic acid, 4-sulfonaphthalene-2,7-dicarboxylic acid.

The copolymers that may be used are those based on isophthalate/sulfoisophthalate, and for example copolymers obtained by condensation of diethylene glycol, cyclohexanedimethanol, isophthalic acid and sulfoisophthalic acid.

The polymers of natural origin, optionally modified, may be chosen from shellac resin, sandarac gum, dammar resins, elemi gums, copal resins and cellulose polymers, and mixtures thereof.

According to a first embodiment of the composition according to the invention, the film-forming polymer may be a water-soluble polymer and may be present in an aqueous phase of the composition; the polymer is thus solubilized in the aqueous phase of the composition.

According to another embodiment variant of the composition according to the invention, the film-forming polymer may be a polymer dissolved in a liquid fatty phase comprising organic solvents or oils such as those described above (the film-forming polymer is thus said to be a liposoluble polymer). The liquid fatty phase may comprise a volatile oil, optionally mixed with a non-volatile oil, the oils possibly being chosen from those mentioned above.

Examples of liposoluble polymers that may be mentioned are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the. vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these dopolymers that may be mentioned are the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Liposoluble film-forming polymers that may also be mentioned include liposoluble copolymers, and for example those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from copolymers of polyvinyl stearate, polyvinyl stearate crosslinked with the aid of divinylbenzene, of diallyl ether or of diallyl phthalate, copolymers of polystearyl(meth)acrylate, polyvinyl laurate and polylauryl(meth)acrylate, it being possible for these poly(meth)acrylates to be crosslinked with the aid of ethylene glycol dimethacrylate or tetraethylene glycol dimethacrylate.

The liposoluble copolymers defined above are known and are described for example in patent application FR-A-2 232 303; they may have a weight-average molecular weight ranging from 2000 to 500 000 and preferably from 4000 to 200 000.

As liposoluble film-forming polymers that may be used in the invention, mention may also be made of polyalkylenes and for example of copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers which may be used in the invention, mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing a type of unit.

Examples of commercially available polymethylsilsesquioxane resins that may be mentioned include those sold:

by the company Wacker under the reference Resin MK, such as Belsil PMS MK, and

by the company Shin-Etsu under the reference KR-220L.

Siloxysilicate resins that may be mentioned include trimethyl siloxysilicate (TMS) resins such as those sold under the reference SR 1000 by the company General Electric or under the reference TMS 803 by the company Wacker. Mention may also be made of the trimethyl siloxysilicate resins sold in a solvent such as cyclomethicone, sold under the name KF-7312J by the company Shin-Etsu, and DC 749 and DC 593 by the company Dow Corning.

Mention may also be made of copolymers of silicone resins such as those mentioned above with polydimethylsiloxanes, for instance the pressure-sensitive adhesive copolymers sold by the company Dow Corning under the reference Bio-PSA and described in document U.S. Pat. No. 5,162,410, or alternatively silicone copolymers derived from the reaction of a silicone resin, such as those described above, and of a diorganosiloxane as described in document WO 2004/073 626.

According to one embodiment of the invention, the film-forming polymer is a film-forming linear block ethylenic polymer, which may comprise at least a first block and at least a second block with different glass transition temperatures (Tg), the said first and second blocks being linked together via an intermediate block comprising at least one constituent monomer of the first block and at least one constituent monomer of the second block.

In one embodiment, the first and second blocks of the block polymer are mutually incompatible.

Such polymers are described, for example, in document EP 1 411 069 or WO 04/028 488.

The film-forming polymer may also be present in the composition in the form of particles dispersed in an aqueous phase or in a non-aqueous solvent phase, which is generally known as a latex or pseudolatex. The techniques for preparing these dispersions are well known to those skilled in the art.

Aqueous dispersions of film-forming polymers that may be used include the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® by the company Interpolymer, Allianz Opt by the company Rohm & Haas, aqueous dispersions of acrylic or styrene/acrylic polymers sold under the brand name Joncryl® by the company Johnson Polymer, or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer; the sulfopolyesters sold under the brand name Eastman AQ® by the company Eastman Chemical Products, vinyl dispersions, for instance Mexomer PAM® from the company Chimex, and mixtures thereof.

As examples of non-aqueous film-forming polymer dispersions, mention may be made of acrylic dispersions in isododecane, for instance MexomerPAP® from the company Chimex, and dispersions of particles of a grafted ethylenic polymer, for example an acrylic polymer, in a liquid fatty phase, the ethylenic polymer for example being dispersed in the absence of additional stabilizer at the surface of the particles as described for example in document WO 04/055 081.

The composition according to the invention may comprise a plasticizer that promotes the formation of a film with the film-forming polymer. Such a plasticizer may be chosen from any compound known to those skilled in the art as being capable of fulfilling the desired function.

Coloring Agent

According to one embodiment, the composition according to the invention may also contain at least one organic or mineral coloring agent, for example of the type such as pigments or nacres.

According to another embodiment, the composition according to the invention may also contain at least one coloring agent chosen from lipophilic dyes, hydrophilic dyes, pigments, nacres and materials with a specific optical effect, and mixtures thereof.

This coloring agent may be present in a proportion of from 0.01% to 50% by weight relative to the total weight of the composition, for example from 0.5% to 40% by weight, for example from 5% to 25%, especially from 0.01% to 20%, for example from 0.1% to 10%, or for example from 2% to 5% by weight, relative to the total weight of the composition.

The term “pigments” should be understood as meaning white or coloured, mineral or organic particles, which are insoluble in an aqueous solution and which are intended to colour and/or opacify the resulting film.

The pigments may be present in a proportion of from 0.01% to 20% by weight, for example from 0.01% to 5% by weight, or from 0.02% to 7% by weight relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

They may also be pigments with a structure that may be, for example, of sericite/brown iron oxide/titanium dioxide/silica type. Such a pigment is sold, for example, under the reference Coverleaf® NS or JS by the company Chemicals and Catalysts, and has a contrast ratio in the region of 30.

The coloring agent may also comprise a pigment with a structure that may be, for example, of silica microsphere type containing iron oxide. An example of a pigment having this structure is the product sold by the company Miyoshi under the reference PC Ball® PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542 669, EP-A-787 730, EP-A-787 731 and WO-A-96/08537.

The term “nacres” should be understood as meaning iridescent or non-iridescent coloured particles of any form, especially produced by certain molluscs in their shell, or else synthesized, and which have a colour effect by optical interference.

The nacres may be chosen from nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic coloring agents.

Examples of nacres that may also be mentioned include natural mica coated with titanium oxide, with iron oxide, with natural pigment or with bismuth oxychloride.

Among the nacres available on the market, mention may be made of the mica-based nacres Timica®, Flamenco® and Duochrome® sold by the company Engelhard, the Timiron® nacres sold by the company Merck, the Prestige® mica-based nacres, sold by the company Eckart, and the Sunshine® synthetic mica-based nacres, sold by the company Sun Chemical.

The nacres may for example have a yellow, pink, red, bronze, orangey, brown, gold and/or coppery colour or tint.

The pigments may or may not be surface-coated, for example surface-treated with silicones, amino acids, fluoro derivatives or any other substance that promotes the dispersion and compatibility of the pigment in the composition.

In an embodiment, the pigments used in the compositions in accordance with the invention may be surface-coated with a lecithin coating. This coating may be obtained by placing a solution of pigment in contact with a lecithin solution, in the presence of divalent or trivalent metal salts. Hydrogenated or non-hydrogenated lecithin may be used to obtain this coating.

The cosmetic composition according to the invention may also comprise water-soluble or liposoluble dyes in a content ranging from 0.01% to 10% by weight, for example ranging from 0.01% to 5% by weight relative to the total weight of the cosmetic composition.

The liposoluble dyes are, for example, Sudan red, DC Red 17, DC Green 6, β-carotene, soybean oil, Sudan brown, DC Yellow 11, DC Violet 2, DC Orange 5 and quinoline yellow.

When the cosmetic compositions according to the invention comprise a water-soluble dye, this dye may be present in the composition in dispersed form.

The cosmetic composition according to the invention may also contain at least one material with a specific optical effect.

This effect is different from a simple conventional hue effect, i.e. a unified and stabilized effect as produced by standard dyestuffs, for instance monochromatic pigments. For the purposes of the invention, the term “stabilized” means lacking an effect of variability of the colour as a function of the angle of observation or alternatively in response to a temperature change.

For example, this material may be chosen from particles with a metallic tint, goniochromatic colouring agents, diffracting pigments, thermochromic agents, optical brighteners, and also fibres, for example interference fibres. Needless to say, these various materials may be combined so as to simultaneously afford two effects, or even a novel effect in accordance with the invention.

The particles with a metallic tint that may be used in the invention may be chosen in particular from:

particles of at least one metal and/or of at least one metal derivative,

particles comprising a mono-material or multi-material organic or mineral substrate, at least partially coated with at least one coat with a metallic tint comprising at least one metal and/or at least one metal derivative, and

mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr and mixtures or alloys thereof (for example bronzes and brasses) are preferred metals.

The term “metal derivatives” is intended to denote compounds derived from metals, especially oxides, fluorides, chlorides and sulfides.

As illustrations of these particles, mention may be made of aluminium particles, such as those sold under the names Starbrite 1200 EAC® by the company Siberline, and Metalureo by the company Eckart.

Mention may also be made of copper metal powders or alloy mixtures such as the reference 2844 sold by the company Radium Bronze, metallic pigments such as aluminium or bronze, such as those sold under the name Rotosafe® 700 from the company Eckart, the silica-coated aluminium particles sold under the name Visionaire Bright Silver® from the company Eckart and metal alloy particles, for instance the silica-coated bronze (alloy of copper and zinc) powders sold under the name Visionaire Bright Natural Gold® from the company Eckart.

They may also be particles comprising a glass substrate, such as those sold by the company Nippon Sheet Glass under the name Microglass Metashine®.

The goniochromatic colouring agent may be chosen, for example, from multilayer interference structures and liquid-crystal colouring agents.

Examples of symmetrical multilayer interference structures that may be used in the compositions prepared in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair by the company Flex; MoS₂/SiO₂/AVSiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by the company Merck (Darmstadt). By way of example, these pigments may be the pigments of silica/titanium oxide/tin oxide structure sold under the name Xirona Magic® by the company Merck, the pigments of silica/brown iron oxide structure sold under the name Xirona Indian Summer® by the company Merck and the pigments of silica/titanium oxide/mica/tin oxide structure sold under the name Xirona Caribbean Blue® by the company Merck. Mention may also be made of the Infinite Colors® pigments from the company Shiseido. Depending on the thickness and the nature of the various layers, different effects are obtained. Thus, with the Fe₂O₃/SiO₂/Al/SiO₂/Fe₂O₃ structure, the colour changes from green-golden to red-grey for SiO₂ layers of 320 to 350 nm; from red to golden for SiO₂ layers of 380 to 400 nm; from violet to green for SiO₂ layers of 410 to 420 nm; from copper to red for SiO₂ layers of 430 to 440 nm.

Examples of pigments with a polymeric multilayer structure that may be mentioned include those sold by the company 3M under the name Color Glitter.

Examples of liquid-crystal goniochromatic particles that may be used include those sold by the company Chenix and also the products sold under the name Helicone® HC by the company Wacker.

The composition of the invention may also comprise any ingredient usually used in the field under consideration.

Needless to say, a person skilled in the art will take care to select the optional additional ingredients and/or the amount thereof such that the advantageous properties of the composition according to the invention are not, or are not substantially, adversely affected by the envisaged addition.

The composition of the invention may be in the form of a coloured lip makeup product, for instance a lipstick, a lip gloss or a lip pencil, optionally having care or treatment properties. It may be in the form of an anhydrous stick.

The composition according to the invention may be manufactured via the known processes generally used in cosmetics or dermatology. For example, it may be manufactured via the following process.

In a first stage, the fillers and pigments may be ground in some of the oily phase.

The rest of the liposoluble ingredients and the phosphate surfactant(s) may then be mixed together at a temperature of about 100° C. The ground material or the predispersed active agents may then be added to the oily phase.

The optional hydrophilic active agents may then be dispersed using a mechanical stirrer.

Finally, the composition may be poured into a mould suitable to give it a dome shape and the whole may then be left to cool to room temperature.

According to another aspect, the invention also relates to a cosmetic assembly comprising:

i) a container delimiting a compartment, the said container being closed by means of a closing member; and

ii) a composition in accordance with the invention, placed inside the said compartment.

The container may be in any adequate form. It may especially be in the form of a bottle, a tube, a jar, a case, a box, a sachet or a carton.

The closing member may be in the form of a removable stopper, a lid, a cap, a tear-off strip or a capsule, especially of the type comprising a body attached to the container and a cover cap articulated on the body. It may also be in the form of a member for selectively closing the container, especially a pump or a valve, for instance a flap valve.

The examples that follow are given as non-limiting illustrations of the present invention.

### EXAMPLES 1 TO 3 OF LIPSTICKS

The compositions of Examples 1 to 3 are obtained according to the following protocol.

In a first stage, the fillers and pigments are ground in some of the oily phase.

The rest of the liposoluble ingredients and the surfactant (cetyl PEG/PPG-10/1-dimethicone, trioleyl phosphate or soybean lecithin) are then mixed together at a temperature of about 100° C. The ground material or the predispersed active agents are then added to the oily phase.

The hydrophilic active agents are then dispersed using a Moritz blender.

Finally, the composition is poured into a mould to give it a dome shape and the whole is left to cool to room temperature.

| | Example 1 | | |
|---|---|---|---|
| | (comparative) | Example 2 | Example 3 |
| | concentration | concentration | concentration |
| Name | (mass %) | (mass %) | (mass %) |
| Polyamide/ | 25 | 25 | 25 |
| polydimethylsiloxane¹ | | | |
| Polyamide/ | 1.2 | 1.2 | 1.2 |
| polydimethylsiloxane² | | | |
| Trioleyl phosphate³ | | 3 | |
| Cetyl PEG/PPG-10/1 | 3 | | |
| Dimethicone⁴ | | | |
| Soybean lecithin⁵ | — | — | 3 |
| Phenyl trimethicone⁶ | 37.9 | 37.9 | 37.9 |
| Polydimethylsiloxane⁷ | 11.58 | 11.58 | 11.58 |
| Octyldodecanol | 0.5 | 0.5 | 0.5 |
| Hydrogenated | 15.68 | 15.68 | 15.68 |
| isoparaffin (6-8 mol | | | |
| of isobutylene)⁸ | | | |
| Titanium oxide | 0.57 | 0.57 | 0.57 |
| D&C Red 7 | 1.1 | 1.1 | 1.1 |
| Black iron oxide | 0.47 | 0.47 | 0.47 |
| Brown, yellow | 1 | 1 | 1 |
| iron oxides | | | |
| Lauroyllysine | 2 | 2 | 2 |

¹DC 2-8179 from Dow Corning
²DC 2-8178 from Dow Corning
³Nikkol TOP-OV from Nikko
⁴Abil EM 90 from Goldschmidt
⁵Topcithin 300 from Degussa
⁶DC 556 from Dow Corning
⁷DC 200 Fluid (10 cSt) from Dow Corning
⁸Parleam from NOF

Comparison of the Sensory Evaluation and the Instrumental Evaluation of the Compositions of Examples 1 to 3

For the sensory evaluation, each composition is applied to the back of the hand of five individuals. The individuals then evaluate the application characteristics and the characteristics of the makeup result on a scale from 0 to 10, with 0 representing the absence of the characteristic and 10 the maximum evaluation.

The value of the parameter attributed to each characteristic corresponds to the mean of the values given for each of the five individuals.

For the instrumental evaluation, the measurements are taken using a TA-XT2i® machine according to the measuring methods described previously.

| | | |
|---|---|---|
| Example 1 | | |
| (comparative) | Example 2 | Example 3 |

SENSORY EVALUATION
ON APPLICATION

| | | | |
|---|---|---|---|
| Glidance | 2.6 | 6.6 | 4.6 |
| Tack | 7.0 | 3.2 | 4.0 |
| Softness | 3.6 | 6.0 | 6.0 |
| Elasticity | 3.8 | 6.0 | 5.6 |

MAKEUP RESULT

| | | | |
|---|---|---|---|
| Film homogeneity | 3.4 | 6.8 | 6.4 |
| Gloss | 4.2 | 7.0 | 7.0 |
| Covering power | 3.6 | 5.2 | 6.0 |

INSTRUMENTAL EVALUATION

| | | | |
|---|---|---|---|
| Hardness (in g) | 166 | 193 | 151 |
| Elasticity (in %) | 97 | 96 | 95 |

It is found that the compositions according to the invention of Examples 2 and 3 have application qualities and a makeup result that are better than those of the comparative Example 1, while at the same time maintaining a hardness and elasticity comparable to or better than those of Example 1.

### EXAMPLE 4

### Lipcare Composition

| | Concentration |
|---|---|
| | mass % |
| Polyamide/polydimethylsiloxane¹ | 25 |
| Polyamide/polydimethylsiloxane² | 1.2 |
| Phenyl trimethicone³ | 34.72 |
| Hydrogenated isoparaffin (6-8 mol of | 14.46 |
| isobutylene)⁴ | |
| Trioleyl phosphate⁵ | 3 |
| Glycerol | 7.5 |
| Lauroyllysine | 2 |
| Polydimethylsiloxane (10 cSt)⁶ | 12.12 |

¹DC 2-8179 from Dow Corning
²DC 2-8178 from Dow Corning
³DC 556 from Dow Corning
⁴Parleam from NOF
⁵Nikkol TOP-0V from Nikko
⁶DC 200 from Dow Corning

This composition may also be pigmented and may contain sodium saccharinate.

This composition gives a glossy, uniform deposit when applied to the lips. It is pleasant to apply. The hardness of this composition is 189 g and the elasticity is 95%.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Cosmetic composition for the lips, comprising, in a physiologically acceptable medium, at least:
one phosphate surfactant, and
one silicone polymer comprising at least one unit comprising:
(1) polyorganosiloxanes comprising at least two groups capable of establishing hydrogen interactions, these two groups being in the polymer chain, and/or
(2) polyorganosiloxanes comprising at least two groups capable of establishing hydrogen interactions, these two groups being on grafts or branches.

2. Composition according to claim 1, in solid form.

3. Composition according to claim 1, with a hardness ranging from 10 to 250 g and/or an elasticity of greater than 80%.

4. Composition according to claim 3, with a hardness ranging from 30 to 200 g.

5. Composition according to claim 3, with a hardness ranging from 50 to 190 g.

6. Composition according to claim 3, with an elasticity of greater than 90%.

7. Composition according to claim 3, with an elasticity close to 95%.

8. Composition according to claim 1, with a total content of silicone polymers representing from 0.5% to 70% of the total weight of the composition.

9. Composition according to claim 1, with a total content of silicone polymers representing from 10% to 45% of the total weight of the composition

10. Composition according to claim 1, in which the said units capable of establishing hydrogen interactions are chosen from ester, amide, sulfonamide, carbamate, thiocarbamate, urea, urethane, thiourea, oxamido, guanidino and biguanidino groups, and combinations thereof.

11. Composition according to claim 1, in which the silicone polymer comprises at least one unit corresponding to the general formula I: 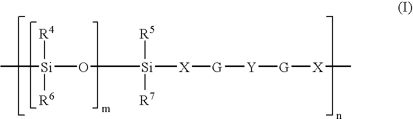
in which:
1) R⁴, R⁵, R⁶ and R⁷, which may be identical or different, represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms;
2) the groups X, which may be identical or different, represent a linear or branched C₁ to C₃₀ alkylenediyl group, possibly containing in its chain one or more oxygen and/or nitrogen atoms;
3) Y is a saturated or unsaturated, C₁ to C₅₀ linear or branched divalent alkylene, arylene, cycloalkylene, alkylarylene or arylalkylene group, possibly comprising one or more oxygen, sulfur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups; or
4) Y represents a group corresponding to the formula: 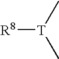
in which
T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
R⁸ represents a linear or branched C₁ to C₅₀ alkyl group or a polyorganosiloxane chain, possibly comprising one or more ester, amide, urethane, thiocarbamate, urea, thiourea and/or sulfonamide groups, which may possibly be linked to another chain of the polymer;
5) the groups G, which may be identical or different, represent divalent groups chosen from: 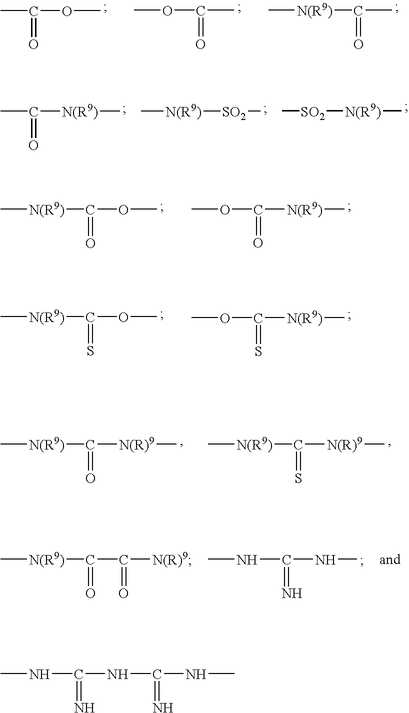
in which R⁹ represents a hydrogen atom or a linear or branched C₁ to C₂₀ alkyl group, on condition that at least 50% of the groups R⁹ of the polymer represent a hydrogen atom and that at least two of the groups G of the polymer are a group other than: 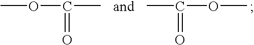
6) n is an integer ranging from 2 to 500, and m is an integer ranging from 1 to 1000.

12. Composition according to claim 11, in which Y represents a group chosen from:
a) linear C₁ to C₂₀ alkylene groups,
b) C₃₀ to C₅₆ branched alkylene groups possibly comprising rings and unconjugated unsaturations,
c) C₅-C₆ cycloalkylene groups,
d) phenylene groups optionally substituted with one or more C₁ to C₄₀ alkyl groups,
e) C₁ to C₂₀ alkylene groups comprising from 1 to 5 amide groups,
f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups, and
g) polyorganosiloxane chains of formula: 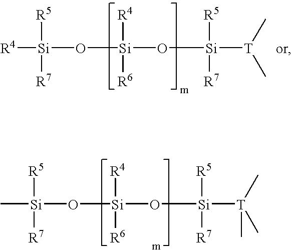
in which.

13. Composition according to claim 1, in which the polymer comprises at least one unit of formula (III) or (IV): 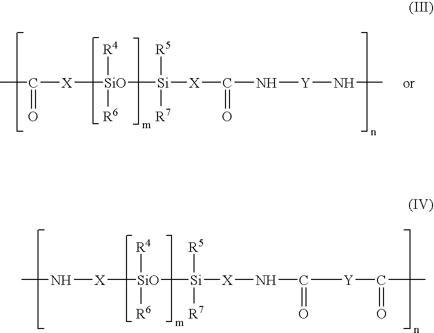
in: R⁴, R⁵, R⁶ and R⁷, which may be identical or different, represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms;
the groups X, which may be identical or different, represent a linear or branched C₁ to C₃₀ alkylenediyl group, possibly containing in its chain one or more oxygen and/or nitrogen atoms;
Y is a saturated or unsaturated, C₁ to C₅₀ linear or branched divalent alylene, arylene, cycloalkylene, alkylarwlene or arylalkylene group, possibly comprising one or more oxygen, sulfur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups: or
Y represents a group corresponding to the formula: 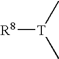
in which
T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
R⁸ represents a linear or branched C₁ to C₅₀ alkyl group or a polvorganosiloxane chain, possibly comprising one or more ester, amide, urethane, thiocarbamate, urea, thiourea and/or sulfonamide grouips, which may possibly be linked to another chain of the polymer;
n is an integer ranging from 2 to 500: and
m is an integer ranging from 1 to 1000.

14. Composition according to claim 13, in which X and/or Y represent(s) an alkylene group containing in its alkylene part at least one of the following components:
1) one to five amide, urea, urethane or carbamate groups,
2) a C₅ or C₆ cycloalkyl group, and
3) a phenylene group optionally substituted with 1 to 3 identical or different C₁ to C₃ alkyl groups,
and/or substituted with at least one component chosen from the group consisting of:
a hydroxyl group,
a C₃ to C₈ cycloalkyl group,
one to three C₁ to C₄₀ alkyl groups,
a phenyl group optionally substituted with one to three C₁ to C₃ alkyl groups,
a C₁ to C₃ hydroxyalkyl group, and
a C₁ to C₆ aminoalkyl group.

15. Composition according to claim 13, in which Y represents: 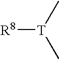
in which R⁸ represents a polyorganosiloxane chain and T represents a group of formula: 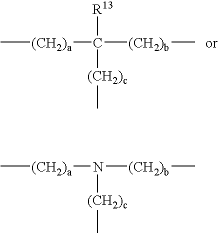
in which a, b and c are, independently, integers ranging from 1 to 10, and R¹³ is a hydrogen atom or a group which may be identical or different, reoresent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms;

16. Composition according to claim 14, in which Y represents: 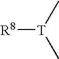
in which R⁸ represents a polyorganosiloxane chain and T represents a group of formula: 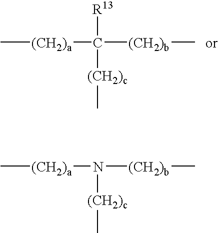
in which a, b and c are, independently, integers ranging from 1 to 10, and R¹³ is a hydrogen atom or a group which may be identical or different, represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms;

17. Composition according to claim 13, in which R⁴, R⁵, R⁶ and R⁷ represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

18. Composition according to claim 14, in which R⁴, R⁵, R⁶ and R⁷ represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

19. Composition according to claim 15, in which R⁴, R⁵, R⁶ and R⁷ represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

20. Composition according to claim 16, in which R⁴, R⁵, R⁶ and R⁷ represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

21. Composition according to claim 1, in which the polymer comprises at least one-unit of formula (VII): 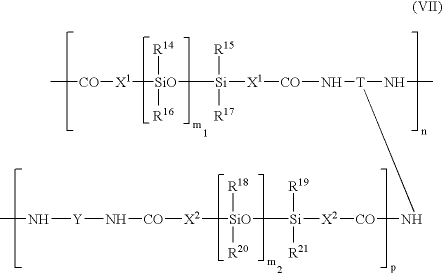
in which: X¹ and X² are identical or different and represent a linear or branched C₁ to C₃₀ alkylenediyl groups possibly containing in its chain one or more oxygen and/or nitrogen atoms;
n is an integer ranging from 2 to 500;
m is an integer ranging from 1 to 1000;
the groups X, which may be identical or different, represent a linear or branched C₁ to C₃₀ alkylenediyl group, possibly containing in its chain one or more oxygen and/or nitrogen atoms;
Y represents a group corresponding to the formula: 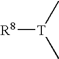
in which
T represents a linear or branched, saturated or unsaturated, C₃ to C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and;
R⁸ represents a linear or branched C₁ to C₅₀ alkyl group or a polyorganosiloxane chain, possibly comprising one or more ester, amide, urethane, thiocarbamate, urea, thiourea and/or sulfonamide groups, which may possibly be linked to another chain of the polymer;
R¹⁴-R²¹ which may be identical or different, represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms,
m₁ and m₂ are numbers in the range from 1 to 1000; and
p is an integer ranging from 2 to 500.

22. Composition according to claim 21, in which:
p is in the range from 1 to 25,
R¹⁴ to R²¹ are methyl groups,
T corresponds to one of the following formulae: 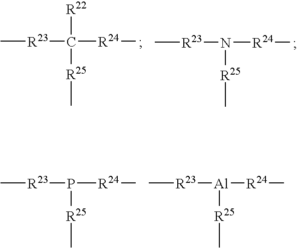
in which R²² is a hydrogen atom or represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms; and
R²³, R²⁴ and R²⁵ are, independently, linear or branched alkylene groups, or of the formula: 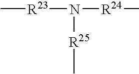
with R²³, R²⁴ and R²⁵ representing —CH₂—CH₂—,
m₁ and m₂ are in the range from 15 to 500,
X₁ and X² represent —(CH₂)₁₀—, and
Y represents —CH₂—.

23. Composition according to claim 1, in which the silicone polymer comprises at least one unit corresponding to formula (VIII) below: 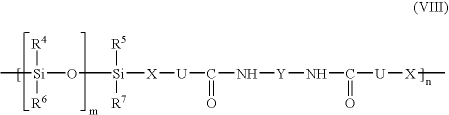
in which: R₄, R₅, R₆ and R₇, which may be identical or different, represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms.
C₆ to C₁₀ aryl groups, optionally substituted with one or more C₁ to C₄ alkyl groups,
polyorganosiloxane chains possibly containing one or more oxygen, sulfur and/or nitrogen atoms;
the groups X, which may be identical or different, represent a linear or branched C₁ to C₃₀ alkylenediyl group, possibly containing in its chain one or more oxygen and/or nitrogen atoms;
Y is a saturated or unsaturated, C₁ to C₅₀ linear or branched divalent alkylene, arylene, cycloalkylene, alkylarylene or arylalkylene group, possibly comprising one or more oxygen, sulfur and/or nitrogen atoms, and/or bearing as substituent one of the following atoms or groups of atoms, fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups; or
Y represents a group corresponding to the formula: 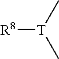
in which
n is an integer ranging from 2 to 500;
m is an integer ranging from 1 to 1000;
U represents —O— or —NH—, such that: 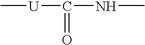
corresponds to a urethane or urea group, or
Y represents a C₅ to C₁₂ cycloaliphatic or aromatic group that may be substituted with a C₁ to C₁₅ alkyl group or a C₅ to C₁₀ aryl group, or Y represents a linear or branched C₁ to C₄₀ alkylene radical or a C₄ to C₁₂ cycloalkylene radical, or Y represents a polyurethane or polyurea block corresponding to the condensation of several diisocyanate molecules with one or more molecules of coupling agents of the diol or diamine type, corresponding to formula (IX): 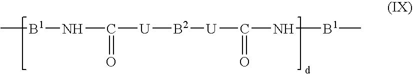
in which B¹ is a group chosen from the groups given above for Y, U is —O— or —NH— and B² is chosen from:
linear or branched C₁ to C₄₀ alkylene groups,
C₅ to C₁₂ cycloalkylene groups, optionally bearing alkyl substituents,
phenylene groups that may optionally bear C₁ to C₃ alkyl substituents, and
groups of formula: 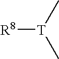
in which T is a hydrocarbon-based trivalent radical possibly containing one or more heteroatoms such as oxygen, sulfur and nitrogen and R⁸ is a polyorganosiloxane chain or a linear or branched C₁ to C₅₀ alkyl chain.

24. Composition according to claim 23, in which Y is a radical chosen from the methylene-4,4-biscyclohexyl radical, the radical derived from isophorone diisocyanate, 2,4- and 2,6-tolylenes, 1,5-naphthylene, p-phenylene and 4,4′-biphenylene-methane.

25. Composition according to claim 1, characterized in that the silicone polymer is chosen from polymers comprising at least one unit corresponding to formula (II): 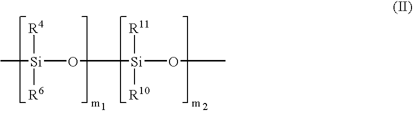
in which
R⁴ and R⁶, which may be identical or different, represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl goups, optionally substituted with one or more C₁ to C₄ alyl groups,
polyorganosiloxane chains possibly containing one or more oxygen, sulfur and/or nitrogen atoms:
R¹⁰ represents a group as defined above for R⁴ and R⁶, or represents a group of formula —X-G-R¹² in which: X, which may be identical or different, represent a linear or branched C₁ to C₃₀ alkylenediyl group, possibly containing in its chain one or more oxygen and/or nitrogen atoms:
G, which may be identical or different, represent divalent groups chosen from: 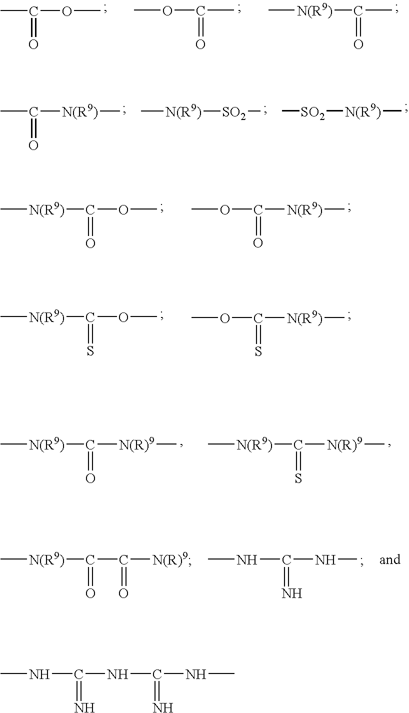
and R¹² represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,
R¹¹ represents a group of formula —X-G-R¹² in which X, G and R¹² are as defined above,
m₁ is an integer ranging from 1 to 998, and
m₂ is an integer ranging from 2 to 500.

26. Composition according to claim 1, comprising at least one polydimethylsiloxane block polymer of general formula (I) as defined in claim 11 and having an index m of greater than 50.

27. Composition according to claim 26, in which n is greater than 100.

28. Composition according to claim 26, in which n is about 120.

29. Composition according to claim 1, comprising at least one first polymer and at least one second polymer, each independently comprising at least one unit of formula (III): 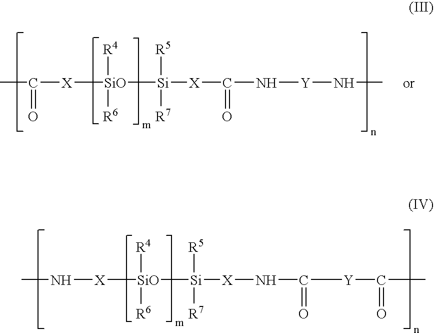
R₄, R₅, R₆ and R₇, which may be identical or different, represent a group chosen from:
linear, branched or cyclic, saturated or unsaturated, C₁ to C₄₀ hydrocarbon-based groups, possibly containing in their chain one or more oxygen, sulfur and/or nitrogen atoms, and possibly being partially or totally substituted with fluorine atoms,
C₆ to C₁₀ aryl groups, optionally substituted with one or more C₁ to C₄ alkyl groups,
polyorganosiloxane chains possibly containing one or more oxygen, sulfur and/or nitrogen atoms,
the groups X, which may be identical or different, represent a linear or branched C₁ to C₃₀ alkylenediyl group, possibly containing in its chain one or more oxygen and/or nitrogen atoms;
Y is a saturated or unsaturated, C₁ to C₅₀ linear or branched divalent alkylene, arylene, cycloallylene, alkylarylene or arylalkylene group, possibly comprising one or more oxygen, sulfur and/or nitrogen atoms, and/or bearing as substituent one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ aminoalkyl groups; or
Y represents a group corresponding to the formula: 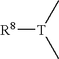
in which
n is an integer ranging from 2 to 500;
m ranges from 50 to 600, for the first polymer, and
m ranges from 5 to 100 for the second polymer.

30. Composition according to claim 29, in which m is about 120 for the first polymer, and m is about 15 for the second polymer.

31. Composition according to claim 29, in which the first polymer has a weight-average molecular mass of between 1000 and 500 000 g/mol.

32. Composition according to claim 29, in which the first polymer has a weight-average molecular mass of between 10 000 and 300 000 g/mol.

33. Composition according to claim 29, in which the second polymer has a weight-average molecular mass ranging from 50 000 to 500 000 g/mol.

34. Composition according to claim 1, in which the phosphate surfactant is chosen from:
(1) the compounds of formula (XIV): 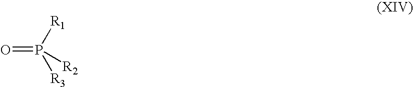
with R₁, R₂ and R₃, which may be identical or different, chosen from:
a group OM with M representing an alkali metal,
a group OR₄, in which R₄ represents a linear, branched, cyclic or aromatic C₅-C₄₀ alkyl group,
an OH group, and
an oxyethylene group (OCH₂CH₂)ₙ(OCH₂CHCH₃)ₘOR with R representing a hydrogen atom or a linear or branched C₁-C₂₀ alkyl group, and n and m being integers with n ranging from 1 to 50 and m ranging from 0 to 50,
(2) glycerophospholipids, and
(3) mixtures thereof.

35. Composition according to claim 34, in which R₁, R₂ and R₃ are identical.

36. Composition according to claim 34, in which at least one, two, or even all the radicals R₁, R₂ and R₃ contain(s) a linear or branched C₁₀-C₃₀ or even C₁₈ alkyl group.

37. Composition according to claim 34, in which R is a C₁₂-C₁₅ alkyl radical.

38. Composition according to claim 34, in which n=10 and/or m=0.

39. Composition according to claim 34, in which the phosphate surfactant comprises trioleyl phosphate, tri-C₁₂₋₁₅ Pareth-10 phosphate or potassium cetyl phosphate.

40. Composition according to claim 34, in which the phosphate surfactant comprises trioleyl phosphate.

41. Composition according to claim 34, in which the glycerophospholipid is defined according to the general formula (XV) below: 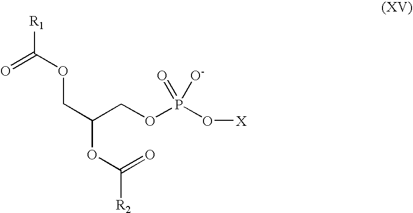
in which:
R₁ and R₂ represent, independently of each other, a saturated or unsaturated, optionally branched fatty acid containing from 4 to 24 carbon atoms, and possibly substituted with one or more hydroxyl and/or amine functions, and
X represents a substituent of general formula R₃R₄R₅N⁺—CH(R₆)—CH₂— in which R₃, R_{4,} R₅ and R₆ represent, independently of each other, a hydrogen atom, alkyl groups containing from 1 to 6 carbon atoms, and/or a carboxyl function.

42. Composition according to claim 41, in which R₁ and R₂, independently of each other, are chosen from butyric acid, caproic acid, caprylic acid, capric acid, caproleic acid, lauric acid, lauroleic acid, myristic acid, myristoleic acid, palmitic acid, palmitoleic acid, stearic acid, oleic acid, linoleic acid, linolenic acid, arachidic acid, isostearic acid, dihydroxystearic acid and ricinoleic acid.

43. Composition according to claim 41, in which X is chosen from choline, serine and/or ethanolamine.

44. Composition according to claim 41, in which the glycerophospholipid comprises phosphatidylcholine, phosphatidylethanolamine and/or phosphatidylserine.

45. Composition according to claim 44, in which the glycerophospholipid is lecithin.

46. Composition according to claim 34, in which the glycerophospholipid is a non-hydrogenated glycerophospholipid.

47. Composition according to claim 1, comprising a nonionic phosphate surfactant.

48. Composition according to claim 1, comprising between 0.1% and 30%, by weight of phosphate surfactant relative to the total weight of the composition.

49. Composition according to claim 1, comprising between 0.5% and 5% by weight of phosphate surfactant relative to the total weight of the composition.

50. Composition according to claim 1, comprising, as phosphate surfactant, trioleyl phosphate and/or lecithin and a polyamide/polydimethylsiloxane and/or a silicone polymer comprising at least one unit of formula (III) as defined in claim 13.

51. Composition according to claim 1, comprising a fatty phase.

52. Composition according to claim 1, comprising a silicone oil.

53. Composition according to claim 1, comprising a hydrocarbon-based oil.

54. Composition according to claim 1, comprising less than 10% of water.

55. Composition according to claim 1, being anhydrous.

56. Composition according to claim 1, also comprising an active agent chosen from:
dermo-relaxing agents,
agents for stimulating the synthesis of dermal or epidermal macromolecules and/or for preventing their degradation,
anti-glycation agents,
anti-irritants,
moisturizers,
desquamating agents,
depigmenting, anti-pigmenting or pro-pigmenting agents,
NO-synthase inhibitors,
agents for stimulating fibroblast or keratinocyte proliferation and/or keratinocyte differentiation,
anti-pollution agents or free-radical scavengers,
soothing agents,
agents acting on the capillary circulation,
agents acting on the energy metabolism of cells,
cicatrizing agents, and
mixtures thereof.

57. Composition according to claim 56, comprising glycerol.

58. Composition according to claim 1, comprising a film-forming polymer.

59. Composition according to claim 1, comprising a coloring agent.

60. Lip makeup process, in which a composition as defined according to claim 1 is applied to the lips.

61. Method for preparing a cosmetic composition for the lips, capable of producing a film of improved gloss comprising the step of combining a silicone polymer as defined in claim 1 in which the phosphate surfactant is chosen from:
(1) the compounds of formula (XIV): 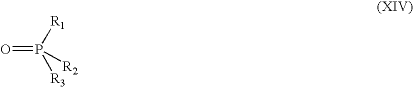
with R₁, R₂ and R₃, which may be identical or different, chosen from:
a group OM with M representing an alkali metal,
a group OR₄, in which R₄ represents a linear, branched, cyclic or aromatic C₅-C₄₀ alkyl group.
an OH group, and
an oxyethylene group (OCH₂CH₂)ₙ(OCH₂CHCH₃)ₘOR with R representing a hydrogen atom or a linear or branched C₁-C₂₀ alkyl group, and n and m being integers with n ranging from 1 to 50 and m ranging from 0 to 50,
(2) glycerophospholipids, and
(3) mixtures thereof.

62. Method for producing a uniform and stabilized dispersion of solid particles, for instance pigments, in a cosmetic composition for the lips comprising the step of combining a silicone polymer as defined in claim 1 in which the phosphate surfactant is chosen from:
(1) the compounds of formula (XIV): 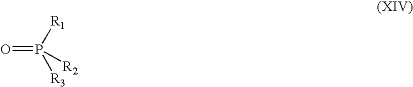
with R₁, R₂ and R₃, which may be identical or different, chosen from:
a group OM with M representing an alkali metal,
a group OR₄, in which R₄ represents a linear, branched, cyclic or aromatic C₅-C₄₀ alkyl group,
an OH group, and
an oxyethylene group (OCH₂CH₂)ₙ(OCH₂CHCH₃)ₘOR with R representing a hydrogen atom or a linear or branched C₁-C₂₀ alkyl group, and n and m being integers with n ranging from 1 to 50 and m ranging from 0 to 50,
(2) glycerophospholipids, and
(3) mixtures thereof.
